(19) United States
(12) Patent Application Publication
(10) Pub. No.: US 2014/0031286 A1
(43) Pub. Date: Jan. 30, 2014

(54) Angiotensin (1-7) Dosage Forms and Uses Thereof

(71) Applicant: University of Southern California, Los Angeles, CA (US)

(72) Inventors: Kathleen E. Rodgers, Long Beach, CA (US); Gere S. DiZerega, San Luis Obispo, CA (US)

(73) Assignee: UNIVERSITY OF SOUTHERN CALIFORNIA, Los Angeles, CA (US)

(21) Appl. No.: 14/022,989

(22) Filed: Sep. 10, 2013

Related U.S. Application Data
(62) Division of application No. 12/400,370, filed on Mar. 9, 2009.
(60) Provisional application No. 61/035,247, filed on Mar. 10, 2008.
(60) Provisional application No. 61/095,052, filed on Sep. 8, 2008.

(51) Int. Cl. A61K 38/08

(52) U.S. Cl. CPC A61K 38/085
USPC 514/9.7

(57) ABSTRACT

The present invention provides angiotensin (1-7) pharmaceutical compositions, dosage forms, and methods for their use, and methods for treating or limiting development of acquired immune deficiency syndrome.

### CROSS REFERENCE

This application claims priority to U.S. Provisional Patent Application Ser. No. 61/035,247 filed Mar. 10, 2008, U.S. Provisional Patent Application Ser. No. 61/095,052 filed Sep. 8, 2008, both incorporated by reference herein in their entirety.

### FIELD OF THE INVENTION

The present invention relates generally to polypeptides, dosage forms, and therapeutic methods.

### BACKGROUND OF THE INVENTION

While the use of angiotensin (1-7) (also referred to herein as “A(1-7)”) for various therapeutic applications has been disclosed, specific dosages to be administered to humans for specific therapeutic outcomes remain uncertain. Such dosage forms and methods for their use would be of use in the art.

The response by the immune system to an immunogen may be depressed as a result of certain diseases or pathological conditions. For example, patients infected with the human immunodeficiency virus (HIV-1) may develop acquired immune deficiency syndrome (AIDS), and thus have depressed immune responses. This patient class is more susceptible to pathological infections or malignancies against which a normal immune system would have otherwise provided sufficient protection.

Current treatments to prevent the development of AIDS in HIV-infected individuals usually involve administration of compounds that inhibit viral DNA synthesis thereby slowing onset of HIV-related immunosuppression and/or administration of protease inhibitors. As these therapies are directed toward anti-retroviral effects, none of the current treatments have proven to be totally effective in treating or preventing development of AIDS. In addition, many of these compounds cause adverse side effects including low platelet count, diarrhea, nausea, renal toxicity, and bone marrow cytopenia (Kempf, et al., U.S. Pat. No. 6,017,928; Lai, et al., U.S. Pat. No. 6,093,743). Thus, there exists a need in the art for improved methods of treating and preventing the development of AIDS and symptoms, disorders, and infections associated with AIDS in HIV-infected patients.

### SUMMARY OF THE INVENTION

In one aspect, the present invention provides pharmaceutical compositions comprising

a) an amount of A(1-7) or a pharmaceutical salt thereof sufficient to provide a dosage to a patient of at least 300 μg/kg; and

b) a pharmaceutically acceptable carrier.

In a further aspect, the present invention provides methods for treating a subject in need of improved immune system function, comprising administering to the subject at least 300 μg/kg/day A(1-7) to provide improved immune system function.

In another aspect, the present invention provides methods and pharmaceutical compositions for treating or limiting acquired immune deficiency syndrome (“AIDS”) development in HIV infected patients, by administering to an HIV-infected subject an amount effective to treat or prevent AIDS development of angiotensinogen, angiotensin I (AI), AI analogues, AI fragments and analogues thereof, angiotensin II (AII), AII analogues, AII fragments or analogues thereof, ACE inhibitors, AII AT₂ type 2 receptor agonists, or agonists of the MAS receptor (Jackson et al, 1988) either alone, combined, or in further combination with other compounds useful for treating immune suppression, including reverse transcriptase inhibitors including but not limited to 3′-azido-3′-deoxythymidine (AZT), 2′,3′-dideoxycytidine (DDC) and 2′,3′-dideoxyinosine (DDI), zidovudine, didanosine, zalcitabine, stavudine, and viramune; protease inhibitors such as Saquinovir™, Nefinavir™, Ritonavir™, and Indinavir™; cytokines such as G-CSF, IL-11, and erythropoietin; and antibiotics or other drugs used for the treatment or prevention of infections in HIV-infected patients.

### BRIEF DESCRIPTION OF THE FIGURES

FIG. 1A is a graph showing A(1-7) treatment effects on patient changes on the mean baseline of CD 34+ cells compared to control.

FIG. 1B is a graph showing A(1-7) treatment effects on patient changes on the mean baseline of CD 34+ cells compared to control on days 8 and 20 after treatment.

FIG. 2A is a graph showing A(1-7) treatment effects on patient changes on the mean baseline of CD 3+ cells compared to control.

FIG. 2B is a graph showing A(1-7) treatment effects on patient changes on the mean baseline of CD 3+ cells compared to control on days 8 and 20 after treatment.

FIG. 3A is a graph showing A(1-7) treatment effects on patient changes on the mean baseline of CD 4+ cells compared to control.

FIG. 3B is a graph showing A(1-7) treatment effects on patient changes on the mean baseline of CD 4+ cells compared to control on days 8 and 20 after treatment.

FIG. 4A is a graph showing A(1-7) treatment effects on patient changes on the mean baseline of CD 8+ cells compared to control.

FIG. 4B is a graph showing A(1-7) treatment effects on patient changes on the mean baseline of CD 8+ cells compared to control on days 8 and 20 after treatment.

### DETAILED DESCRIPTION OF THE INVENTION

In one aspect, the present invention provides pharmaceutical compositions, comprising

a) an amount of a peptide consisting of at least 5 amino acids of angiotensin (1-7) (“A(1-7)”) or a pharmaceutical salt thereof sufficient to provide a dosage to a patient of 300 μg/kg or more; and

b) a pharmaceutically acceptable carrier.

As is known in the art, “A(1-7)” is a peptide having the amino acid sequence Asp-Arg-Val-Tyr-Ile-His-Pro. In various embodiments, the peptide consists of Asp-Arg-Val-Tyr-Ile (A(1-5)), Asp-Arg-Val-Tyr-Ile-His (A(1-6)), or A(1-7).

The inventors have discovered that human administration of doses of A(1-7) of 300 μg/kg or more in the clinical studies reported herein provides an unexpected benefit by increasing the number of various white blood cell types (such as lymphocytes) in peripheral blood above baseline levels, while lower doses (ie: 200 μg/kg) did not provide such an increase over baseline. Thus, the pharmaceutical compositions of the inventions are useful, for example, in prophylactic and therapeutic treatment of subjects that can benefit from increased while blood cells in peripheral blood, as described in more detail below.

In various embodiments, the amount of A(1-7) or pharmaceutical salt thereof is sufficient to provide a dosage to a patient of between 300 μg/kg and 10 mg/kg; 300 μg/kg and 5 mg/kg; 300 μg/kg and 1000 μg/kg; 300 μg/kg and 900 μg/kg 300 μg/kg and 900 μg/kg; 300 μg/kg and 800 μg/kg; 300 μg/kg and 700 μg/kg; 300 μg/kg and 600 μg/kg; 300 μg/kg and 500 μg/kg; or 300 μg/kg and 400 μg/kg.

In a preferred embodiment of this first aspect of the invention, the pharmaceutical composition comprises an A(1-7) dosage form of between 1.5 mg and 1000 mg of A(1-7) or a pharmaceutical salt thereof. In various further preferred embodiments, the amount of A(1-7) or pharmaceutical salt thereof in the dosage form is between 2 mg and 1000 mg; 2 mg and 750 mg; 5 mg and 750 mg; 10 mg and 1000 mg; 2 mg and 500 mg; 2 mg and 250 mg; 5 mg and 500 mg; 10 mg and 750 mg, 10 mg and 500 mg, 15 mg and 1000 mg, 10 mg and 250 mg, 10 mg and 100 mg, 15 mg and 750 mg, 15 mg and 500 mg, 15 mg and 250 mg, and 15 mg and 100 mg. In preferred embodiments for once a day administration, the dosage form comprises 10 mg-1000 mg A(1-7) or pharmaceutical salt thereof; in various further preferred embodiments, between 10 mg and 750 mg, 10 mg and 500 mg, 10 mg and 250 mg, 15 mg and 1000 mg, 15 mg and 750 mg, 15 mg and 500 mg, 15 mg and 250 mg; 15 mg and 200 mg; 15 mg and 150 mg; 15 mg and 100 mg; 15 mg and 75 mg; and 15 mg and 50 mg. In preferred embodiments for twice a day administration, the dosage form comprises 5 mg and 500 mg, 5 mg and 150 mg, 5 mg and 100 mg, 5 mg and 50 mg, 7.5 mg and 500 mg, and 7.5 mg-250 mg A(1-7) or pharmaceutical salt thereof; in various further preferred embodiments for twice a day administration, between 7.5 mg and 125 mg; 7.5 mg and 100 mg; 7.5 mg and 75 mg; 7.5 mg and 50 mg; 7.5 mg and 37.5 mg; and 7.5 mg and 25 mg. It is well within the level of skill in the art, based on the teachings herein, to determine appropriate dosage forms for administration more than twice per day.

Suitable acids which are capable of forming salts with A(1-7) include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming salts with A(1-7) include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

The pharmaceutical compositions of the invention may be made up in a solid form (including granules, powders or suppositories) or in a liquid form (e.g., solutions, suspensions, or emulsions). The pharmaceutical compositions may be applied in a variety of solutions. Suitable solutions for use in accordance with the invention are sterile, dissolve sufficient amounts of the A(1-7), and are not harmful for the proposed application. In this regard, the compounds of the present invention are very stable but are hydrolyzed by strong acids and bases. The compounds of the present invention are soluble in organic solvents and in aqueous solutions at pH 5-8.

The pharmaceutical compositions may be subjected to conventional pharmaceutical operations such as sterilization and/or may contain conventional adjuvants, such as preservatives, stabilizers, wetting agents, emulsifiers, buffers etc.

The pharmaceutical compositions of the invention can be prepared to be administered by any suitable route, including but not limited to subcutaneous, intradermal, transdermal (for example, by slow-release polymers), intramuscular, intraperitoneal, intravenous, oral, aural, epidural, anal or vaginal (for example, by suppositories), and intranasal routes, infusion or bolus injection, or absorption through epithelial or mucocutaneous linings.

In a preferred embodiment, the pharmaceutical composition is prepared for administration by the subcutaneous route. In one preferred embodiment for subcutaneous administration, the A(1-7) or salt thereof may comprise from 0.0001% to 10% w/w; in one embodiment, not more than 5% w/w, and in a further preferred embodiment from 0.1% to 2% w/w of the formulation. In any of these embodiments, the amount of A(1-7) or pharmaceutical salt thereof is preferably sufficient to provide a dosage to a patient of between 300 μg/kg and 1000 μg/kg; 300 μg/kg and 900 μg/kg 300 μg/kg and 900 μg/kg; 300 μg/kg and 800 μg/kg; 300 μg/kg and 700 μg/kg; 300 μg/kg and 600 μg/kg; 300 μg/kg and 500 μg/kg; or 300 μg/kg and 400 μg/kg; and/or preferably sufficient to provide the preferred dosage forms disclosed above.

In another embodiment, the A(1-7) or salt thereof is prepared as a stable lyophilized peptide formulation that can be reconstituted with a suitable diluent to generate a reconstituted pharmaceutical compositions of the invention that are suitable for subcutaneous administration When reconstituted with a diluent comprising a preservative (such as bacteriostatic water for injection), the reconstituted formulation may be used as a multi-use formulation. Such a formulation is useful, for example, where the subject requires frequent subcutaneous administrations of A(1-7). The advantage of a multi-use formulation is that it facilitates ease of use for the patient, reduces waste by allowing complete use of vial contents, and results in a significant cost savings for the manufacturer since several doses are packaged in a single vial (lower filling and shipping costs). Such reconstituted formulations would also be suitable for use with other types of parenteral administration.

For administration, the pharmaceutical compositions are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In another embodiment of any of the above embodiments, the pharmaceutical composition may further comprise an amount effective of a cytokine for increasing hematopoietic cell production. In various embodiments, the cytokine may be selected from the group consisting of granulocyte colony stimulating factor, granulocyte-macrophage-colony stimulating factor (GM-CSF), epidermal growth factor, interleukin 11, thrombopoietin, megakaryocyte development and growth factor, pixykines, stem cell factor, FLT (fms-like tyrosine kinase)-ligand, and interleukins 1, 3, 6, and 7.

In other embodiments, the pharmaceutical compositions of the present invention may further comprise one or more other therapeutics as needed by a given subject.

In another embodiment of the pharmaceutical compositions of the first aspect of the invention, the pharmaceutical composition is loaded in a drug delivery device. This embodiment provides for ease of use whether in a clinical setting, or where the subject can self-administer in their home. In various embodiments, the drug delivery device may be selected from the group consisting of syringes, injection devices (e.g. the Inject-Ease™ and Genject™ devices), injector pens (such as the GenPen™) needleless devices (e.g. MediJector™ and BioJector™), and subcutaneous patch delivery systems.

A(1-7) or salts thereof can further be derivatized to provide enhanced plasma half-life, for example, by linking to polyethylene glycol. The A(1-7) or salts thereof may comprise L-amino acids, D-amino acids (which are resistant to L-amino acid-specific proteases in vivo), a combination of D- and L-amino acids, and various “designer” amino acids (e.g., β-methyl amino acids, Cα-methyl amino acids, and Nα-methyl amino acids, etc.) to convey special properties. Synthetic amino acids include norleucine for isoleucine.

In addition, the A(1-7) or salts thereof can have peptidomimetic bonds. For example, an A(1-7) peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such polypeptides are resistant to protease activity, and possess an extended half-live in vivo.

A(1-7) or salts thereof may be chemically synthesized or recombinantly expressed, each of which can be accomplished using standard methods in the art.

In a second aspect, the present invention provides methods for treating a human subject in need of improved immune system function, comprising administering to the human subject A(1-7) at a dosage of at least 300 μg/kg/day to provide improved immune system function. The inventors have discovered that human administration of at least 300 μg/kg/day A(1-7) in the clinical study detailed below provides an unexpected benefit by increasing the number of various white blood cell types in peripheral blood above baseline levels, while lower doses (ie: 200 μg/kg/day) did not provide such an increase over baseline. Thus, the pharmaceutical compositions of the inventions are useful, for example, in prophylactic and therapeutic treatment of human subjects that can benefit from increased while blood cells in peripheral blood. Thus, in a various embodiments, the methods may comprise treatment with any of the pharmaceutical compositions of the first aspect of the invention.

As used herein, “improved immune system function” is an increase in one or more white blood cell types as a result of treatment relative to a baseline level of the one or more white blood cell types in the absence of such treatment (for example, no treatment or treatment with a control). In various embodiments, the increase in one or more white blood cell types relative to the subject's baseline white blood cell count comprises increases in lymphocytes. In a further preferred embodiment, the increase in lymphocytes comprises increases in one or more of CD4, CD8 and CD3 cells relative to baseline.

An “increase in baseline” is any increase that may be of value in immune system function in the subject; in one embodiment the increase is statistically significant; in other embodiments, the increase is at least 5%, 10%, 20%, 35%, 40%, 50%, 60%, 70%, 80%, 90%, 100%, or more over baseline.

As described in the examples below, clinical results on normal subjects have demonstrated a dose-related change in the number of certain lymphocytes. At the dose of 300 μg/kg/day (but not 200 μg/kg/day), there was an approximately 15-20% increase in CD3 (pan T cell marker) during study drug administration that rapidly diminished after cessation of treatment. A similar increase in CD4+ and CD8+ cells was observed during the treatment period.

The human subject can be any subject that can benefit from improved immune system function. As such, the treatment can be prophylactic or therapeutic. In various non-limiting examples, the subject may be (a) suffering from a tumor (and can benefit from an improved immune system function to aid in natural host defenses against a tumor); (b) undergoing or scheduled to undergo chemotherapy (and thus may benefit from any improvement in immune system function); (c) suffering from an infection, such as a viral infection; including but not limited to HIV infection (d) immunodeficient due to conditions including, but not limited to aging, malnutrition, drug addiction, alcoholism, genetic predisposition, antibiotic treatment, skin damage, and certain disease states such as diabetes and AIDS; (e) at increased risk of exposure/infection by one or more infectious agents (e.g.: health care workers, first responders to emergencies, military personnel, hospitalized patients, subjects exposed to pathogenic infectious agent but not yet showing symptoms of infection, etc.).

In one non-limiting example, the subject is infected with an infectious agent, such as HIV, and thus in need of amelioration or elimination of the infectious agent. In the case of HIV infection (or other immunocompromised states), the methods of the invention can be used to limit development of or treat opportunistic infections (infections with an organism that would not normally be pathologic in patients with intact immune systems), such as Aspergillus, Candida albicans, and Kaposi's sarcoma.

In another non-limiting example, populations at high risk for HIV infection include but are not limited to partners of HIV infected individuals, sex trade workers, health care workers, and intravenous drug users, and such subjects can benefit from treatment by the methods of the invention.

The dosage may be administered prior to, at the same time, or subsequent to a particular treatment or exposure as a prophylactic treatment. The A(1-7) or salts thereof can be administered by any suitable route, including but not limited to subcutaneous, intradermal, transdermal (for example, by slow-release polymers), intramuscular, intraperitoneal, intravenous, oral, aural, epidural, anal or vaginal (for example, by suppositories), and intranasal routes, infusion or bolus injection, or absorption through epithelial or mucocutaneous linings. In one preferred embodiment, the A(1-7) or salts are administered subcutaneously.

Thus, the subject may, for example, receive the recited dosage between 1, 2, or 3 times per day prior to or during periods of treatment/exposure and then receive the same dosage, a lower dosage or no dosage subsequent to treatment/exposure. In one embodiment, subcutaneous administration of between about 300 to 1000 μg/kg/day of the active agents is initiated at between one week before to one week after administration of a chemotherapeutic agent.

A(1-7) or salts thereof may be administered as the sole treatment or in conjunction with other drugs or therapies useful in treating the condition in question.

In another aspect, the present invention provides methods and pharmaceutical compositions, for treating or limiting acquired immune deficiency syndrome (“AIDS”) development in HIV infected patients, by administering to an HIV-infected patient an amount effective to treat or limit AIDS development of a polypeptide comprising or consisting of angiotensinogen, angiotensin I (AI), AI analogues, AI fragments and analogues thereof, angiotensin II (AII), AII analogues, AII fragments or analogues thereof, ACE inhibitors, AII AT₂ type 2 receptor agonists, or agonists of the MAS receptor, either alone, combined, or in further combination with other compounds useful for treating immune suppression, including reverse transcriptase inhibitors including but not limited to 3′-azido-3′-deoxythymidine (AZT), 2′,3′-dideoxycytidine (DDC) and 2′,3′-dideoxyinosine (DDI), zidovudine, didanosine, zalcitabine, stavudine, and viramune; protease inhibitors such as Saquinovir™ Nefinavir™, Ritonavir™, and Indinavir™; cytokines such as G-CSF, IL-11, and erythropoietin; and antibiotics or other drugs used for the treatment or prevention of infections in HIV-infected patients.

Unless otherwise indicated, the term “angiotensin converting enzyme inhibitors” or “ACE inhibitors” includes any compound that inhibits the conversion of the decapeptide angiotensin Ito angiotensin II, and includes but is not limited to alacepril, alatriopril, altiopril calcium, ancovenin, benazepril, benazepril hydrochloride, benazeprilat, benzazepril, benzoylcaptopril, captopril, captopril-cysteine, captopril-glutathione, ceranapril, ceranopril, ceronapril, cilazapril, cilazaprilat, converstatin, delapril, delapril-diacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentoprili, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat. (See for example Jackson, et al., Renin and Angiotensin in Goodman & Gilman's The Pharmacological Basis of Therapeutics, 9th ed., eds. Hardman, et al. (McGraw Hill, 1996); and U.S. Pat. No. 5,977,159.)

As used herein, the term “HIV” includes all variants and types of HIV-1, HIV-2, and other synonymous retroviruses, such as human T-lymphotropic virus type III (HTLV-III) and lymphadenopathy associated virus (LAV-1 and LAV-2).

As used herein, the term “AIDS” refers to acquired immune deficiency syndrome, AIDS-related complex (ARC), and diminution in lymphocyte numbers in HIV-infected individuals.

As used herein, the term “treating or limiting AIDS” includes limiting immunosuppression caused by AIDS and reducing or decreasing the rate of immunosuppression caused by AIDS, a as well as limiting or reducing the associated symptoms, disorders, and infections associated with HIV infection, including but not limited to susceptibility to pathogenic and opportunistic organisms and infections, anemia, thrombocytopenia, and lymphopenia. While not being bound by any mechanism of action, such effects may be accomplished by, for example, decreasing HIV levels in the patient's peripheral blood lymphocytes, by increasing lymphocyte numbers; by replenishing the bone marrow; and/or by increasing survival of HIV-infected patients;

As used herein, the term “opportunistic infection” refers to infections with an organism that would not normally be pathologic in patients with intact immune systems.

As hereinafter defined, a preferred class of AT2 agonists for use in accordance with the present invention comprises AII, AII analogues or active fragments thereof having p-NH-Phe in a position corresponding to a position 6 of AII. In addition to peptide agents, various non-peptidic agents (e.g., peptidomimetics) having the requisite AT2 agonist activity are further contemplated for use in accordance with the present invention.

The active AII analogues, fragments of AII and analogues thereof of particular interest in accordance with the present invention comprise or consist of a sequence of at least three contiguous amino acids of groups R¹-R⁸ in the sequence of general formula I

R¹-R²-R³-R⁴-R⁵-R⁶-R⁷-R⁸ (SEQ ID NO: 4)
wherein R¹ is selected from the group consisting of H, Asp, Glu, Asn, Acpc (1-aminocyclopentane carboxylic acid), Ala, Me²Gly, Pro, Bet, Glu(NH₂),

Gly, Asp(NH₂) and Suc, or is absent,
R² is selected from the group consisting of Arg, Lys, Ala, Cit, Orn, Ser(Ac), Sar, D-Arg and D-Lys,
R³ is selected from the group consisting of Val, Ala, Leu, norLeu, Ile, Gly, Lys, Pro, Aib, Acpc and Tyr;
R⁴ is selected from the group consisting of Tyr, Tyr(PO₃)₂, Thr, Ser, homoSer, azaTyr, and Ala;
R⁵ is selected from the group consisting of Ile, Ala, Leu, norLeu, Val and Gly;
R⁶ is selected from the group consisting of His, Arg or 6-NH₂-Phe;
R⁷ is selected from the group consisting of Pro or Ala; and
R⁸ is selected from the group consisting of Phe, Phe(Br), Ile and Tyr,
excluding sequences including R⁴ as a terminal Tyr group.

Compounds falling within the category of AT2 agonists useful in the practice of the invention include the AII analogues set forth above subject to the restriction that R⁶ is p-NH₂-Phe.

In a further preferred embodiment of each of the above embodiments,

R¹ is selected from the group consisting of Asp and Glu, or is absent;

R² is selected from the group consisting of Arg, Lys, and Ala;

R³ is selected from the group consisting of Val, Ala, Leu, norLeu, Ile, Gly, Lys, and Pro;

R⁴ is selected from the group consisting of Tyr and homoSer;

R⁵ is selected from the group consisting of Ile, Ala, Leu, norLeu, Val and Gly;

R⁶ is selected from the group consisting of His and Arg;

R⁷ is selected from the group consisting of Pro or Ala; and

R⁸ is selected from the group consisting of Phe, Ile, or is absent.

In alternate embodiments, the polypeptides comprise or consist of at least four, five, six, seven, or eight contiguous amino acids of groups R¹-R⁸ in the sequence of general formula I. In a further alternative, the polypeptides consists of a sequence of at least four, five, six, seven, or eight contiguous amino acids of groups R¹-R⁸ in the sequence of general formula I.

Particularly preferred combinations for R¹ and R² are Asp-Arg, Asp-Lys, Glu-Arg and Glu-Lys. Particularly preferred embodiments of this class include the following: AIII or AII(2-8), Arg-Val-Tyr-Ile-His-Pro-Phe [SEQ ID NO:5]; AII(3-8), also known as des 1-AIII or AIV, Val-Tyr-Ile-His-Pro-Phe [SEQ ID NO:6]; AII(1-7), Asp-Arg-Val-Tyr-Ile-His-Pro [SEQ ID NO:7]; AII(2-7). Arg-Val-Tyr-Ile-His-Pro [SEQ ID NO:8]; AII(3-7), Val-Tyr-Ile-His-Pro [SEQ ID NO:9]; AII(5-8), Ile-His-Pro-Phe [SEQ ID NO:10]; AII(1-6), Asp-Arg-Val-Tyr-Ile-His [SEQ ID NO:11]; AII(1-5), Asp-Arg-Val-Tyr-Ile [SEQ ID NO:12]; AII(1-4), Asp-Arg-Val-Tyr [SEQ ID NO:13]; and AII(1-3), Asp-Arg-Val. Other preferred embodiments include: Arg-norLeu-Tyr-Ile-His-Pro-Phe [SEQ ID NO:15] and Arg-Val-Tyr-norLeu-His-Pro-Phe [SEQ ID NO:16]. Still another preferred embodiment encompassed within the scope of the invention is a peptide having the sequence Asp-Arg-Pro-Tyr-Ile-His-Pro-Phe [SEQ ID NO:17]. AII(6-8), His-Pro-Phe and AII(4-8), Tyr-Ile-His-Pro-Phe [SEQ ID NO: 19] were also tested and found not to be effective.

Other preferred embodiments comprise or consist of

SEQ ID NO: 20
Asp-Arg-Val-Tyr-Val-His-Pro-Phe
SEQ ID NO: 21
Asn-Arg-Val-Tyr-Val-His-Pro-Phe
SEQ ID NO: 22
Ala-Pro-Gly-Asp-Arg-Ile-Tyr-Val-His-Pro-Phe
SEQ ID NO: 23
Glu-Arg-Val-Tyr-Ile-His-Pro-Phe
SEQ ID NO: 24
Asp-Lys-Val-Tyr-Ile-His-Pro-Phe
SEQ ID NO: 25
Asp-Arg-Ala-Tyr-Ile-His-Pro-Phe
SEQ ID NO: 26
Asp-Arg-Val-Thr-Ile-His-Pro-Phe
SEQ ID NO: 27
Asp-Arg-Val-Tyr-Leu-His-Pro-Phe
SEQ ID NO: 28
Asp-Arg-Val-Tyr-Ile-Arg-Pro-Phe
SEQ ID NO: 29
Asp-Arg-Val-Tyr-Ile-His-Ala-Phe
SEQ ID NO: 30
Asp-Arg-Val-Tyr-Ile-His-Pro-Tyr
SEQ ID NO: 31
Pro-Arg-Val-Tyr-Ile-His-Pro-Phe
SEQ ID NO: 32
Asp-Arg-Pro-Tyr-Ile-His-Pro-Phe
SEQ ID NO: 33
Asp-Arg-Val-Tyr(PO₃)₂-Ile-His-Pro-Phe
SEQ ID NO: 34
Asp-Arg-norLeu-Tyr-Ile-His-Pro-Phe
SEQ ID NO: 35
Asp-Arg-Val-Tyr-norLeu-His-Pro-Phe
SEQ ID NO: 36
Asp-Arg-Val-homoSer-Tyr-Ile-His-Pro-Phe

Another class of polypeptides of particular interest in accordance with the present invention are those of the general formula II:

R²-R³-R⁴-R⁵-R⁶-R⁷-R⁸ (SEQ ID NO: 37)
in which R² is selected from the group consisting of H, Arg, Lys, Ala, Orn, Citron, Ser(Ac), Sar, D-Arg and D-Lys; and
R³-R⁸ are as defined above.

In the above formulas, the standard three-letter abbreviations for amino acid residues are employed. In the absence of an indication to the contrary, the L-form of the amino acid is intended. Other residues are abbreviated as is known in the art:

Analogues of particular interest include the following:

SEQ ID NO: 20

| | |
|---|---|
| Analogue 1 | Asp-Arg-Val-Tyr-Val-His-Pro-Phe |

SEQ ID NO: 21

| | |
|---|---|
| Analogue 2 | Asn-Arg-Val-Tyr-Val-His-Pro-Phe |

SEQ ID NO: 22

| | |
|---|---|
| Analogue 3 | Ala-Pro-Gly-Asp-Arg-Ile-Tyr-Val- |
| | His-Pro-Phe |

SEQ ID NO: 23

| | |
|---|---|
| Analogue 4 | Glu-Arg-Val-Tyr-Ile-His-Pro-Phe |

SEQ ID NO: 24

| | |
|---|---|
| Analogue 5 | Asp-Lys-Val-Tyr-Ile-His-Pro-Phe |

SEQ ID NO: 25

| | |
|---|---|
| Analogue 6 | Asp-Arg-Ala-Tyr-Ile-His-Pro-Phe |

SEQ ID NO: 26

| | |
|---|---|
| Analogue 7 | Asp-Arg-Val-Thr-Ile-His-Pro-Phe |

SEQ ID NO: 27

| | |
|---|---|
| Analogue 8 | Asp-Arg-Val-Tyr-Leu-His-Pro-Phe |

SEQ ID NO: 28

| | |
|---|---|
| Analogue 9 | Asp-Arg-Val-Tyr-Ile-Arg-Pro-Phe |

SEQ ID NO: 29

| | |
|---|---|
| Analogue 10 | Asp-Arg-Val-Tyr-Ile-His-Ala-Phe |

SEQ ID NO: 30

| | |
|---|---|
| Analogue 11 | Asp-Arg-Val-Tyr-Ile-His-Pro-Tyr |

SEQ ID NO: 31

| | |
|---|---|
| Analogue 12 | Pro-Arg-Val-Tyr-Ile-His-Pro-Phe |

SEQ ID NO: 32

| | |
|---|---|
| Analogue 13 | Asp-Arg-Pro-Tyr-Ile-His-Pro-Phe |

SEQ ID NO: 33

| | |
|---|---|
| Analogue 14 | Asp-Arg-Val-Tyr(PO3)2-Ile-His-Pro- |
| | Phe |

SEQ ID NO: 34

| | |
|---|---|
| Analogue 15 | Asp-Arg-norLeu-Tyr-Ile-His-Pro-Phe |

SEQ ID NO: 35

| | |
|---|---|
| Analogue 16 | Asp-Arg-Val-Tyr-norLeu-His-Pro-Phe |

SEQ ID NO: 36

| | |
|---|---|
| Analogue 17 | Asp-Arg-Val-homoSer-Tyr-Ile-His- |
| | Pro-Phe |

Other particularly preferred embodiments include:

| | | | |
|---|---|---|---|
| 1GD | Al4-AII(1-7) | DRVAIHP | SEQ ID NO: 38 |
| 2GD | Pro3-AII(1-7) | DRPYIHP | SEQ ID NO: 39 |
| 5GD | Lys3-AII(1-7) | DRKYIHP | SEQ ID NO: 40 |
| 9GD | NorLeu-AII(1-7) | DR(nor)YIHP | SEQ ID NO: 41 |
| GSD 28 | Ile⁸-AII | DRVYIHPI | SEQ ID NO: 42 |
| | Ala3aminoPhe6 AII: | RVAIHPF | SEQ ID NO: 43 |
| | Ala3-AIII | RVAIHPF | SEQ ID NO: 44 |
| | Gly¹-AII | GRVYIHPF | SEQ ID NO: 45 |
| | NorLeu⁴-AIII | --RVYnLHPF | SEQ ID NO: 46 |
| | Acpc³-AII | DR(Acpc)YIHPF | SEQ ID NO: 47 |
| GSD 37B | Orn²-AII | D(Orn)VYIHPF | SEQ ID NO: 48 |
| GSD38B | Citron²-AII | D(Citron)VYIHPF | SEQ ID NO: 49 |
| 3GD | Pro³Ala⁴-AII(1-7) | DRPAIHP | SEQ ID NO: 50 |
| 8GD | Hydroxy-Pro³-AII(1-7) | DRP(OH)AIHP | SEQ ID NO: 51 |

The polypeptides of the instant invention may be synthesized by any conventional method, including, but not limited to, those set forth in J. M. Stewart and J. D. Young, Solid Phase Peptide Synthesis, 2nd ed., Pierce Chemical Co., Rockford, Ill. (1984) and J. Meienhofer, Hormonal Proteins and Peptides, Vol. 2, Academic Press, New York, (1973) for solid phase synthesis and E. Schroder and K. Lubke, The Peptides, Vol. 1, Academic Press, New York, (1965) for solution synthesis. The disclosures of the foregoing treatises are incorporated by reference herein.

In general, these methods involve the sequential addition of protected amino acids to a growing peptide chain (U.S. Pat. No. 5,693,616, herein incorporated by reference in its entirety). Normally, either the amino or carboxyl group of the first amino acid and any reactive side chain group are protected. This protected amino acid is then either attached to an inert solid support, or utilized in solution, and the next amino acid in the sequence, also suitably protected, is added under conditions amenable to formation of the amide linkage. After all the desired amino acids have been linked in the proper sequence, protecting groups and any solid support are removed to afford the crude polypeptide. The polypeptide is desalted and purified, preferably chromatographically, to yield the final product.

Preferably, peptides are synthesized according to standard solid-phase methodologies, such as may be performed on an Applied Biosystems Model 430A peptide synthesizer (Applied Biosystems, Foster City, Calif.), according to manufacturer's instructions. Other methods of synthesizing peptides or peptidomimetics, either by solid phase methodologies or in liquid phase, are well known to those skilled in the art. Alternatively, the peptides may be produced via conventional molecular biological methods.

In one aspect of the present invention methods for treating or limiting AIDS in an HIV-infected patient, comprising administering to an HIV-infected patient an amount effective to treat or limit AIDS of at least one compound selected from angiotensinogen, AI, AI analogues, and/or AI fragments and analogues thereof, AII, AII analogues, AII fragments and analogues thereof, AII AT₂ type 2 receptor agonists, ACE inhibitors, and/or agonists of the MAS receptor (hereinafter referred to as the “active agent”), alone, in combination with each other, or in combination with other compounds that are beneficial for treating or preventing AIDS in HIV-infected individuals, including but not limited to reverse transcriptase inhibitors including but not limited to 3′-azido-3′-deoxythymidine (AZT), 2′,3′-dideoxycytidine (DDC) and 2′,3′-dideoxyinosine (DDI), zidovudine, didanosine, zalcitabine, stavudine, and viramune; protease inhibitors such as Saquinovir™, Nefinavir™, Ritonavir™, and Indinavir™; cytokines such as G-CSF, IL-11, and erythropoietin; and antibiotics or other drugs used for the treatment or prevention of infections in HIV-infected patients.

In one embodiment, the HIV-infected patient is one that is undergoing HAART (highly active antiretroviral therapy). In another embodiment, the HIV-infect patient has a CD4+ T-lymphocyte counts less than 200/mm³, where the method comprises improving CD4+ T-lymphocyte recovery in the patient (ie: to reduce or eliminate lymphopenia in the patients). In a further embodiment, the methods of the invention result in CD4+ T-lymphocyte counts greater than 350/mm³.

While not bound by any specific mechanism, the inventors believe that administration of the active agents to HIV-infected individuals provides for recovery from or improvement in symptoms and effects of HIV infection or the effects of medication. Such therapeutic efficacy may be manifested in numerous ways, including but not limited to increases in immune cell populations such as CD4+ cells, platelets (such as when the patient presents with thrombocytopenia as a result of drug treatment or the disease), or red blood cells (anemia as a result of drug treatment or the disease).

For use in inducing in treating or limiting development of AIDS in an HIV-infected individual, the active agents may be administered by any suitable route, but are preferably administered either orally, parentally, by inhalation spray, transdermally, intravenously, rectally, intra-arterially, or topically in dosage unit formulations containing conventional pharmaceutically acceptable carriers, adjuvants, and vehicles. The term parenteral as used herein includes subcutaneous, intramuscular, intravenously, intra-arterially, or intratendinous. In one embodiment, polypeptides are modified to facilitate oral delivery, such as by lipidization of the polypeptide or other modifications to allow the polypeptides to bypass gastric enzymes.

The active agent may also be administered directly to the HIV-infected individual in a pharmaceutically suitable vehicle, for example, a solution of 5% DMSO or 10% ethanol in saline. In a preferred embodiment, multiple administrations of the active agents are made over the period of time encompassing effective treatment.

A large variety of alternatives are known in the art as suitable for purposes of sustained release and are contemplated as within the scope of the present invention. Suitable delivery vehicles include, but are not limited to, the following: microcapsules or microspheres; liposomes and other lipid-based release systems; crystalloid and viscous instillates; absorbable and/or biodegradable mechanical barriers; and polymeric delivery materials, such as polyethylene oxide/polypropylene oxide block copolymers (e.g. poloxamers), poly-orthoesters, cross-linked polyvinyl alcohol, polyanhydrides, polymethacrylate and polymethacryladmide hydrogels, anionic carbohydrate polymers, etc. Useful delivery systems are well known in the art and are described in, e.g., U.S. Pat. No. 4,937,254, the entire disclosure of which is hereby incorporated by reference.

The active agent may be made up in a solid form (including granules, powders or suppositories) or in a liquid form (e.g., solutions, suspensions, or emulsions), and may be subjected to conventional pharmaceutical operations such as sterilization and/or may contain conventional pharmaceutically acceptable adjuvants, such as stabilizers, wetting agents, emulsifiers, preservatives, cosolvents, suspending agents, viscosity enhancing agents, ionic strength and osmolality adjustors and other excipients in addition to buffering agents. Suitable water soluble preservatives which may be employed in the drug delivery vehicle include sodium bisulfite, sodium thiosulfate, ascorbate, benzalkonium chloride, chlorobutanol, thimerosal, phenylmercuric borate, parabens, benzyl alcohol, phenylethanol or antioxidants such as Vitamin E and tocopherol and chelators such as EDTA and EGTA. These agents may be present, generally, in amounts of about 0.001% to about 5% by weight and, preferably, in the amount of about 0.01 to about 2% by weight.

Suitable acids which are capable of forming salts with the polypeptides include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming salts with A(1-7) include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

For administration, the active agent is ordinarily combined with one or more pharmaceutically acceptable adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In various embodiments, the amount of peptide or pharmaceutical salt thereof is sufficient to provide a dosage to a patient of between 100 μg/kg and 10 mg/kg; 100 μg/kg and 5 mg/kg; 100 μg/kg and 1000 μg/kg; 200 μg/kg and 10 mg/kg; 200 μg/kg and 5 mg/kg; 200 μg/kg and 1000 μg/kg; 300 μg/kg and 10 mg/kg; 300 μg/kg and 5 mg/kg; 300 μg/kg and 1000 μg/kg; or any other dosage ranges as disclosed for the first and second aspect of the invention. In a preferred embodiment of all of the other embodiments of this aspect of the invention, the active agent comprises or consists of 5, 6, or 7 amino acids of A(1-7); most preferably comprising or consisting of A(1-7).

The active agents can be administered as often as deemed appropriate by an attending physician based on all relevant factors. In one embodiment, administration is once per day; in another, dosing is twice per day.

The efficacy of the active agents are determined by methods that measure indications such as decreases in HIV levels in the patient's peripheral blood lymphocytes, viral load, anemia, thrombocytopenia, and lymphopenia; and increased CD4+ cell counts, lymphocyte numbers, antibody titer, resistance to pathogenic and opportunistic infections, and survival of HIV-infected patients.

The active agents of the present invention may also be administered in a further stabilized form, such as, for example, associated with polyethylene glycol or as a fusion protein, or other forms known in the art.

In another aspect of the invention, pharmaceutical compositions are provided that comprise an amount effective for treating or limiting AIDS in an HIV-infected individual of one or more of the active agents of the present invention. In a preferred embodiment the pharmaceutical compositions also comprise one or more other compounds that are useful for treating or preventing AIDS in an HIV-infected individual, including but not limited to 3′-azido-3′-deoxythymidine (AZT), 2′,3′-dideoxycytidine (DDC) and 2′,3′-dideoxyinosine (DDI), zidovudine, didanosine, zalcitabine, stavudine, and viramune; and protease inhibitors such as Saquinovir™ Nefinavir™, Ritonavir™, and Indinavir™.

The present invention may be better understood with reference to the accompanying examples that are intended for purposes of illustration only and should not be construed to limit the scope of the invention.

### Example 1

### A Study of the Safety, Tolerability and Effects on Peripheral Blood Cell Counts of A(1-7) Administered Subcutaneously Once Daily for 7 Days in Healthy Men

### Methodology:

Single-center, randomized, double-blind, placebo-controlled, 2 cohort dose-escalation trial of once daily dosing over a 7-day period (7 doses total):
   Cohort 1: 2 subjects treated with placebo and 8 subjects treated with 200 μg/kg USB003 (A(1-7))
   Cohort 2: 2 subjects treated with placebo and 8 subjects treated with 300 μg/kg USB003
For each cohort, there was a Screening Period, Treatment Phase, Follow-up Period, and Study Termination or Early Withdrawal Day. Subjects were confined to the study unit during the Treatment Phase.
Cohort 2 was treated approximately 1 week after completion of the Treatment Phase for Cohort 1 to allow time for the assessment of safety and tolerability in the first cohort.

### Number of Subjects:

Twenty-one healthy male subjects (15 Caucasian, 4 Black, 1 Hispanic, and 1 “other”) between 22 and 43 years of age, inclusive.

### Diagnosis and Main Criteria for Inclusion:

To qualify for study participation, subjects must have: been healthy male subjects; been aged 18 to 45 years old, inclusive; had a body mass index (BMI) within the range of 19 to 29; had negative tests for drugs of abuse including cannabinoids, alcohol, opiates, cocaine, amphetamines, and benzodiazepines; had a cotinine value below 400 ng/mL; had negative tests for human deficiency virus (HIV) and hepatitis C virus (HCV) antibodies; and must have had a negative test for hepatitis B surface antigen.

### Test Product, Dose, Mode of Administration:

A(1-7) (also referred to herein as USB003), sterile solution, 45 mg/mL, single dose vials, single SC injection once daily for 7 days at 200 (Cohort 1) or 300 μg/kg (Cohort 2).

### Duration of Treatment:

The study duration for each cohort was up to 13 days of screening; followed by SC doses of test drug or placebo for 7 days; and safety assessments for 20 days.

### Reference Therapy, Dose, Mode of Administration:

Matched placebo, the individual components were identical to those in the USB003 product, with the exception of the active ingredient angiotensin 1-7 (A 1-7); single SC injection once daily for 7 days.

### Criteria for Evaluation:

### Safety:

Safety and tolerance evaluations included:
Tolerability including local reactions at the injection site.
Vital signs (blood pressure [BP], pulse rate [PR], respiration rate [RR], and temperature).
Routine clinical laboratory tests (hematology, serum biochemistry, and urinalysis).
Recording of adverse events (AE).
Electrocardiograms (ECG) at baseline and Day 8.

### Pharmacological Effect:

The following evaluations (under efficacy and safety in the protocol) were performed:
Effects on the number of mature blood cells:
   1. CBC (with differential), platelet count, reticulocyte count.
Effects on early hematopoietic progenitor cells and mature lymphocyte subsets in the periphery:
   1. Flow cytometry to evaluate frequency of peripheral blood progenitor cells (eg, CD34⁺) and lymphocyte subsets (eg, CD3, CD4, CD8) in peripheral blood.
   2. Collection of blood for later possible culture for progenitor cells (eg, colony forming units-granulocyte/macrophage [CFU-GM]) to evaluate potential effects on CD34⁺ hematopoietic progenitor cells in peripheral blood.
   3.

### Statistical Methods:

Descriptive statistics were used to summarize adverse events (AEs). Descriptive statistics, with change from baseline, were used for clinical laboratory results, vital signs, and flow cytometry data. Shift tables were prepared for clinical laboratory results. Other data are listed.

### Summary of Results and Conclusions

### Pharmacological Effect:

During the administration of USB003, there were elevations in the peripheral blood concentrations of WBC, neutrophils, CD3, CD34+, CD4 and CD8. All hematological parameters returned to baseline within 12 days following discontinuation of USB003 administration.

### Pharmacokinetics:

Samples were collected but have not been analyzed at this time.

### Safety Results:

In general, USB003, at SC doses of 200 and 300 μg/kg was safe and well tolerated.
There were no deaths or serious adverse events (SAE), and no subject discontinued from the study because of an AE.
Five subjects (4 in the 300 μg/kg and 1 in placebo dose group) did have elevations in their oral body temperature high enough to be assessed as an AE. These were all assessed as possibly related to study drug. They all had a Common Toxicity Criteria (CTC) score of Grade 1, but were otherwise asymptomatic.
Adverse events, other than increased temperature, that were assessed as possibly related to study drug were increased alanine aminotransferase (ALT), headache, and cystic nodule on anterior abdominal wall.
Adverse events that occurred during the study, whose relationship to study drug was assessed as doubtful, included increased ALT and aspartate aminotransferase (AST), headache, acid reflux, WBCs in the urine and glucose in urine.
Upper respiratory infection, increased body temperature, increased ALT, increased WBC, and headache were each reported in more than 1 subject.
There were no AEs with CTC scores of Grade 3 or higher and only 1 Grade 2 toxicity.
Three subjects had 1 AE each that required treatment with concomitant medication (increased temperature, acid reflux, and upper respiratory infection).
There was an increase in creatinine clearance observed at the 200 μg/kg dose level but it did not correlate with a decrease in serum creatinine and was not observed in the 300 μg/kg dose level.
A slight increase in the 24-hour urinary excretion of sodium was observed in both treatment groups.
All AEs resolved by the Study Termination (except ankle sprain), and there were no changes in clinical laboratory tests, 12-lead ECGs, vital signs, or PEs that were of continuing clinical concern.

### Conclusions:

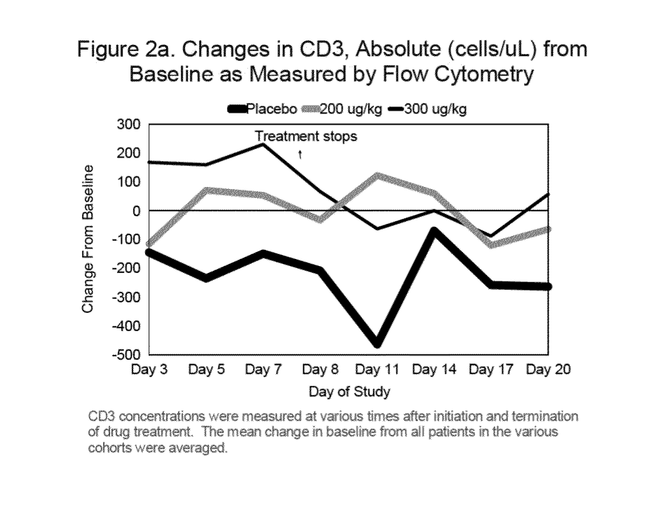

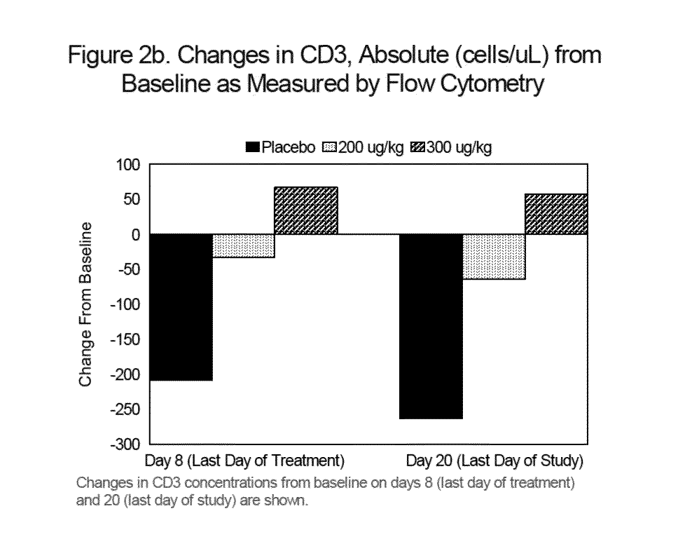

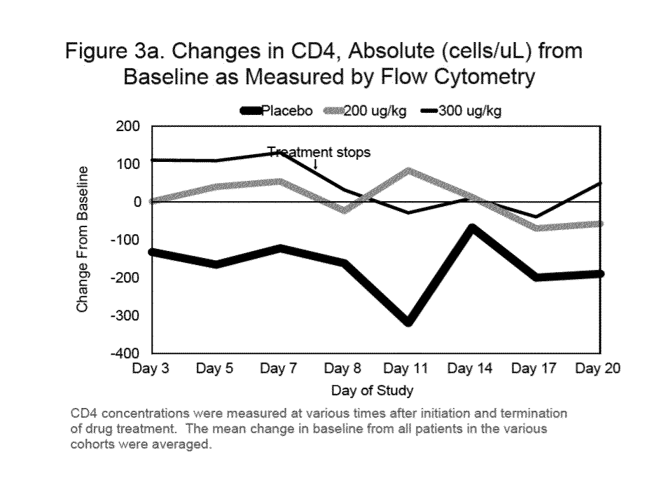

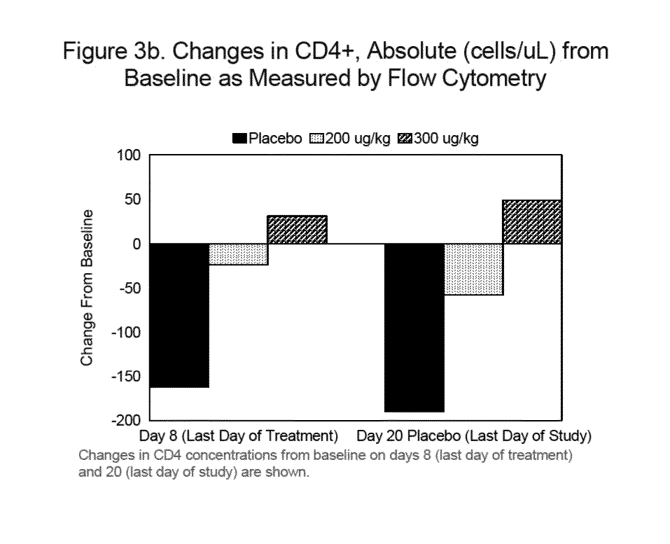

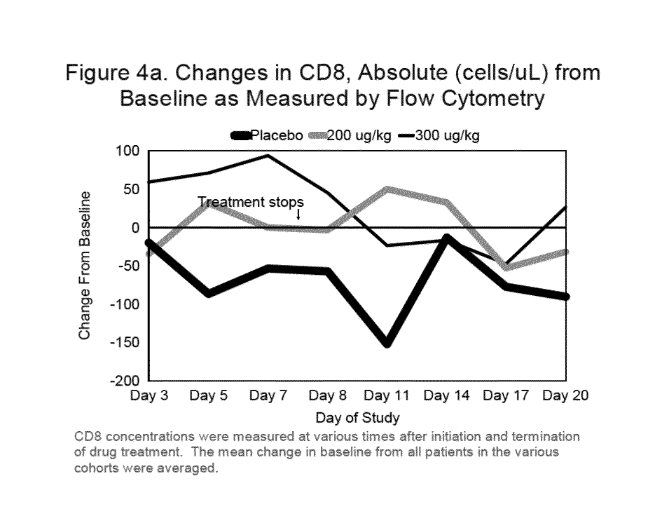

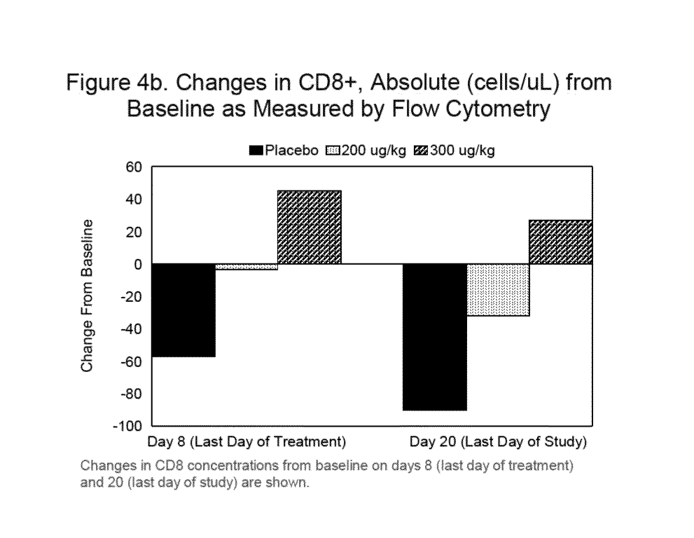

USB003 was safe and well tolerated at both the 200 and 300 μg/kg SC doses. There were no SAEs, and no AE led to withdrawal from the study. Increased body temperature and isolated liver enzyme elevations were the only AEs that occurred in more than one subject administered USB003.
USB003 had an effect on hematologic parameters. The findings were increases in total WBC and neutrophil concentrations. In addition, concentrations of CD3, CD34+, CD4 and CD8 were increased during USB003 administration then recovered to baseline levels within 10 days following discontinuation of therapy.
At the dose of 300 μg/kg/day (but not at 200 ug/kg/day), there was an approximately 15-20% increase in CD3 (pan T cell marker) during study drug administration that rapidly diminished after cessation of treatment (FIG. 2). A similar increase in CD4+ and CD8+ cells was observed during the treatment period (FIGS. 3 and 4).

### Overall Study Design and Plan: Description

This was a single-center, double-blind, randomized, placebo-controlled, dose-escalation study of once daily subcutaneous (SC) dosing over a 7-day period (7 doses total) of USB003. Two cohorts of 10 subjects each were treated in the study as described below:
Cohort 1: 2 subjects treated with placebo and 8 subjects treated with 200 μg/kg USB003.
Cohort 2: 2 subjects treated with placebo and 8 subjects treated with 300 μg/kg USB003.

The subjects were confined to the clinic during the Treatment Phase. The drug was administered daily by SC injection. The subject, Investigator, and the Investigator's support staff were blinded (except for the staff pharmacist) as to the treatment group. Dosing in the second cohort was initiated approximately 1 week after completion of the Treatment Phase for Cohort 1, to allow time for the assessment of safety and tolerability in the first cohort. Selected safety parameters and blood samples for hematology and clinical chemistry measurements were collected for an additional 12-day period after the Treatment Phase of each group.

The study consisted of Screening, Check-in (Day −1), Treatment (Days 1 through 7), Checkout (Day 8), and Follow-up Visits (Days 11, 14, 17, and 20). During Screening, baseline evaluations consisted of a medical history, physical examination (PE), vital sign and temperature measurements, and laboratory testing, including hematology and chemistry panels, urinalysis, and urine toxicology screen for drugs of abuse. Screening tests for human immune deficiency virus (HIV) as well as hepatitis C virus (HCV) antibodies and hepatitis B surface antigen were also performed. These baseline evaluations were to establish that the subject was healthy and met entry criteria.

On Day −1, subjects who met the eligibility criteria underwent a PE, vital sign measurements, and had samples drawn to evaluate clinical laboratory parameters. On Study Days 1 through 7 for each cohort, subjects received a single SC injection of USB003 at the assigned dose level or placebo. Subjects were kept in the clinic under observation for the entire Day −1 to Day 8 period. Subjects were discharged from the clinic on the morning of Day 8 after all scheduled procedures and a 24-hour urine collection had been completed. Subjects were scheduled to return to the clinic on Days 11, 14, 17, and 20 for hematology measurements and selected safety assessments. Subjects were to be released from the study following a post-study PE on Day 20.
Two placebo and 8 active drug subjects at the 200 μg/kg level were treated first. When no dose-limiting adverse events (AE) occurred within 7 days of completion of the Treatment Phase of cohort 1, the study proceeded and patients were entered on the next dose level. If a dose-limiting AE had occurred within 7 days of the last dose, the decision to proceed to the next level would have been based upon the review of the safety data and its relationship to the study drug by the Investigator and the Sponsor. Dose-limiting effects were defined as any Grade 3 toxicity in any subject or the frequent occurrence of Grade 2 toxicities. If dose-limiting effects had occurred in subjects receiving USB003 in the first dosage cohort, no dose escalation would have been performed, and the study would have been stopped, pending medical review by the Investigator and the Sponsor. The Investigator and Sponsor were to be blinded during this process. New subjects were enrolled for treatment at the 300 μg/kg level. In this cohort, 2 placebo and 8 active drug subjects were treated. The same dose-limiting evaluation procedures, as detailed above, were applied to the subjects in Cohort 2 (300 μg/kg level).

Safety assessments during the study period included vital sign measurement (systolic [SBP] and diastolic [DBP] blood pressure, heart rate [HR], respiratory rate [RR] in the sitting position, and oral temperature), clinical chemistry, hematology, urinalysis, 12-lead electrocardiogram (ECG) in the supine position, and AE monitoring. Clinical chemistry/hematology samples were drawn within a 30-minute period prior to dosing on selected dosing days.

### Screening Phase

Subjects were screened for enrollment within 13 days prior to Day −1 of the study. Subjects were screened in accordance with predefined entrance criteria. The following procedures were completed during the Screening Period:
Complete medical history;
Complete PE, including height, weight, ECG, temperature, and vital sign measurements;
Documentation of any clinically significant preexisting conditions;
Laboratory profile;
Urine toxicology screen for cannabinoids, opiates, cocaine, amphetamines, benzodiazepines, alcohol, and cotinine; and
HIV and HCV antibodies, hepatitis B surface antigen.

### Treatment Phase

### Day −1

Subjects who had qualified for the study reported to the clinic by 6:30 AM the day prior to dosing for baseline clinical laboratory (including hematology for flow cytometry) evaluations, PE, vital signs, body weight, urine toxicology screen, 24-hour urine collection (for creatinine clearance), and baseline USB003 plasma concentration measurements. In addition, hematology, serum chemistry, and urinalysis were performed. The clinical laboratory results were available to the Investigator prior to dosing the morning of Day 1.

### Day 1

On Day 1, prior to drug administration, vital signs, body weight, and blood samples for clinical laboratory evaluations were taken. If the number of CD34⁺ cells in a subject's sample exceeded 10 cells/μL in the flow cytometry hematology panel, an additional blood sample was to have been collected on the next day, and at subsequent scheduled hematology assessments to undergo analysis using a second panel consisting of additional markers to identify subsets of CD34⁺ cells.

Fasting of subjects prior to dosing was not required. USB003 was administered SC in the abdomen by a qualified study staff member. A physician was present at dosing, and available during the observation period for this day and all days that study drug was administered.

Vital signs were measured immediately prior to dosing, every hour for the first 6 hours post-dosing, then every 3 hours up to 12 hours post-dosing.

A standard lunch was allowed at 4 hours after dosing.

### Days 2-6

Prior to drug administration, vital signs (on Days 2 through 6) and blood samples were taken within 5 minutes before dosing (on Days 2, 3, 4, and 5). Hematology profiles were measured on Days 3 and 5 just prior to dosing. If the number of CD34⁺ cells in a subject's sample ever exceeded 10 cells/μL in the flow cytometry hematology panel, an additional blood sample was to have been collected on the next day and at subsequent scheduled hematology assessments to undergo analysis using a second panel consisting of additional markers to identify subsets of CD34⁺ cells.

Blood biochemistry and urinalysis were performed on Day 4, just prior to dosing. Fasting of subjects prior to dosing was not required. USB003 was administered SC in the abdomen by a qualified study staff member at the same time each day. Vital signs were measured at 1, 3 and 6 hours post-dosing.

A standard lunch was allowed at 4 hours after dosing.

### Day 7

On Day 7, prior to drug administration, vital signs, and blood samples for hematology parameters were measured. If the number of CD34⁺ cells exceeded 10 cells/μL in the flow cytometry hematology panel, an additional blood sample was to have been collected from that subject on the next day and at subsequent scheduled hematology assessments to undergo analysis using a second panel consisting of additional markers to identify subsets of CD34⁺ cells.

Urine was collected over a 24-hour period (beginning at approximately 8 μm) for determination of creatinine clearance. USB003 was administered SC in the abdomen by a qualified study staff member 24 hours after the previous (Day 6) injection.

Vital signs were measured immediately prior to dosing on Day 7, then at 1, 3, and 6 hours following dosing.

A standard lunch was allowed at 4 hours after dosing.

### Day 8

On Day 8, PE, vital signs, body weight, ECG, blood for hematology and biochemistry, as well as a urine sample for urinalysis were taken. If the number of CD34⁺ cells exceeded 10 cells/μL in the flow cytometry hematology panel, an additional blood sample was to have been collected from that subject on the next day and at subsequent scheduled hematology assessments to undergo analysis using a second panel consisting of additional markers to identify subsets of CD34⁺ cells. The subject was discharged from the unit upon completion of these procedures, and completion of the 24-hour urine collection.

### Follow-Up Period

Subjects were to have returned to the clinic on Days 11, 14, and 17. On Day 11, blood was drawn for plasma USB003 and hematology measurement, and on Days 14 and 17, blood was drawn for hematology only. If the number of CD34⁺ cells exceeded 10 cells/μL in the flow cytometry hematology panel, an additional blood sample was to be collected on the next day and at subsequent scheduled hematology assessments to undergo analysis using a second panel consisting of additional markers to identify subsets of CD34⁺ cells.

### Study Termination

On Day 20 of the study, subjects were to have returned to the clinic for the post-study check and PE. Blood samples for hematology and plasma USB003 were also drawn.

### Evaluations for Pharmacological Activity

The effect of USB003 on the number of mature blood cells was assessed by the complete blood count with differential, platelet and reticulocyte counts. The effects on early hematopoietic progenitor cells and mature lymphocyte subsets in the periphery were assessed by flow cytometry. In addition, blood was collected and processed to enable culture of progenitors at a later time.

### Safety Evaluations

Safety evaluations are based on AEs, PEs, vital signs, temperature, and laboratory results from baseline to study completion. Laboratory measurements were conducted by a local clinical laboratory. Using standard procedures, blood samples were drawn or urine collected from subjects for the following laboratory tests:

Hematology Profile

Red blood cell (RBC) count, RBC indices, white blood cell count (WBC) with differential, reticulocyte count, platelet count, hemoglobin (Hb), hematocrit (Hct), plasma hemoglobin, peripheral flow cytometry, including CD34⁺, flow cytometry for enumeration of CD3, CD4, and CD8 lymphocytes, and culture for progenitor cells in peripheral blood including colony-forming units-granulocyte/macrophage (CFU-GM).

Clinical Chemistry

Aspartate aminotransferase (AST), alanine aminotransferase (ALT), gamma, glutamyltranferase (GGT), creatinine, creatine kinase, sodium, potassium, bicarbonate (CO₂), chloride, glucose, magnesium, calcium, inorganic phosphorous, blood urea nitrogen (BUN), total protein, albumin, total bilirubin, direct and indirect bilirubin, uric acid, and alkaline phosphatase,

Urinalysis with Microscopic Examination of Sediment

Blood, urobilinogen, ketones, glucose, protein, microscopic examination of sediment, sodium, and potassium.

Urine Toxicology Screen (at Screen and on Day −1)

Creatinine Clearance
Pooled 24 hour urine samples were collected from approximately 8 a.m. on Day −1 to 8 a.m. on Day 1 and from 8 a.m. on Day 7 to 8 a.m. on Day 8 for creatinine measurement and calculation of creatinine clearance

HIV and HCV Antibody Testing and Hepatitis B Surface Antigen Testing (at Screen Only)

The protocol describes the number of mature blood cells and the effects on early hematopoietic progenitor cells and mature lymphocyte subsets in the periphery as efficacy parameters. The outcome of these analyses is below.

While the protocol uses the term baseline to mean the pre-dose measurements on each study day, the analysis plan defines baseline as the last measurable value prior to dosing on Study Day 1. Therefore, instead of the descriptive statistics measuring the changes from baseline to post-dose on each day (a matter of a few hours), the time interval between these measured time points was days.

### Study Subjects

### Disposition of Subjects

Subject disposition is summarized in Table 1. One subject withdrew consent after 3 days of dosing and was replaced with a subject using the same randomized treatment assignment.

TABLE 1
Subject Disposition

| | Number (%) of Subjects | | | |
|---|---|---|---|---|
| | | USB003 | USB003 | |
| | Placebo | 200 μg/kg | 300 μg/kg | Overall |
| Total Number | 4 | 9 | 8 | 21 |
| Randomized | | | | |
| Total Number | 4 (100.0) | 8 (88.9) | 8 (100.0) | 20 (95.2) |
| Completed | | | | |
| Total Number | 0 (0.0) | 1 (11.1) | 0 (0.0) | 1 (4.8) |
| Discontinued | | | | |
| Withdrew Consent | 0 (0.0) | 1 (11.1) | 0 (0.0) | 1 (4.8) |

The listings classify the subjects by screening number. A conversion of this screening number to the appropriate randomization number and dose group is presented in Table 2.

TABLE 2
Subject Randomization and Screening Number and Dose Group

| Randomization | Screening | |
|---|---|---|
| Number | Number | Dose Group |
| 01 | 003 | Placebo |
| 02 | 006 | USB003 200 μg/kg |
| 03 | 007 | USB003 200 μg/kg |
| 04 | 009 | USB003 200 μg/kg |
| 05 | 011* | USB003 200 μg/kg |
| 06 | 012 | USB003 200 μg/kg |
| 07 | 017 | Placebo |
| 08 | 025 | USB003 200 μg/kg |
| 09 | 027 | USB003 200 μg/kg |
| 10 | 028 | USB003 200 μg/kg |
| 11 | 046 | USB003 300 μg/kg |
| 12 | 042 | USB003 300 μg/kg |
| 13 | 043 | USB003 300 μg/kg |
| 14 | 041 | Placebo |
| 15 | 048 | USB003 300 μg/kg |
| 16 | 053 | USB003 300 μg/kg |
| 17 | 052 | USB003 300 μg/kg |
| 18 | 055 | USB003 300 μg/kg |
| 19 | 060 | Placebo |
| 20 | 045 | USB003 300 μg/kg |
| 55 | 033 | USB003 200 μg/kg |

*Withdrew consent after 3 doses

### Pharmacokinetic and Efficacy Evaluations

### Data Sets Analyzed

One population, the Safety Population, was used for all summaries and analyses of study data. The Safety Population is defined as all subjects who received at least one dose of study drug and had at least one post-dose safety or tolerability assessment.

### Demographic and Other Baseline Characteristics

As summarized in Table 3, the study population consisted of all male subjects. The population was 71.4% Caucasian, 19.0% Black, 4.8% Hispanic and 4.8% Other. The mean (SD) height was 177.6 (8.05) cm, weight 82.1 (10.33) kg, and Body Mass Index (BMI) was 26.0 (2.24) kg/m². The demographic and baseline characteristics were similar among dose groups, except for race, where there was a notably higher percentage of Caucasians in the placebo and 200 μg/kg groups.

At baseline (the last measurable value prior to dosing on Day 1), the mean SBP and DBP were similar for the 3 dosing groups. The mean SBP was 117.3, 119.8, and 120.1 mm Hg for the placebo, USB003 (200 μg/kg), and USB003 (300 μg/kg) dose groups, respectively. The mean DBP at baseline was 76.5, 78.7, and 78.8 mm Hg for the placebo, USB003 (200 μg/kg), and USB003 (300 μg/kg) dose groups, respectively. The mean baseline oral body temperature was similar among the dose groups (range=36.3 to 36.5° C.), however, the mean baseline oral body temperature (for which all changes from baseline are calculated for the entire study) for all 3 groups was approximately 1° C. lower than the generally accepted average mean oral body temperature for normal subjects. The placebo, 200 μg/kg, and 300 μg/kg USB003 dose group's baseline mean respiratory rates were similar 14.5-15.8 breaths/min); however, the mean HR in the placebo group (75.0 beats/min) was higher than either the 200 μg/kg (64.6 beats/min) or the 300 μg/kg (68.0 beats/min) groups.

TABLE 3
Demographic and Baseline Characteristics

| | | | USB003 | USB003 | |
|---|---|---|---|---|---|
| | | Placebo | 200 μg/kg | 300 μg/kg | Overall |
| Age (years) | N | 4 | 9 | 8 | 21 |
| | Mean | 29.0 | 34.1 | 33.3 | 32.8 |
| | SD | 4.76 | 7.24 | 7.34 | 6.85 |
| | Median | 27.0 | 37.0 | 31.0 | 30.0 |
| | Min, Max | 26.0, 36.0 | 24.0, 43.0 | 22.0, 43.0 | 22.0, 43.0 |
| Race | Caucasian | 3 (75.0) | 8 (88.9) | 4 (50.0) | 15 (71.4) |
| | Black | 1 (25.0) | 0 (0.0) | 3 (37.5) | 4 (19.0) |
| | Hispanic | 0 (0.0) | 1 (11.1) | 0 (0.0) | 1 (4.8) |
| | Other | 0 (0.0) | 0 (0.0) | 1 (12.5) | 1 (4.8) |
| Gender | Male | 4 (100.0) | 9 (100.0) | 8 (100.0) | 21 (100.0) |
| Height (cm) | N | 4 | 9 | 8 | 21 |
| | Mean | 177.8 | 176.3 | 179.0 | 177.6 |
| | SD | 3.77 | 9.26 | 8.75 | 8.05 |
| | Median | 176.5 | 178.0 | 178.0 | 178.0 |
| | Min, Max | 175.0, 183.0 | 157.0, 191.0 | 163.0, 191.0 | 157.0, 191.0 |
| Screening | N | 4 | 9 | 8 | 21 |
| Weight (kg) | Mean | 80.0 | 82.3 | 83.0 | 82.1 |
| | SD | 4.08 | 10.44 | 13.03 | 10.33 |
| | Median | 81.5 | 80.0 | 81.5 | 81.0 |
| | Min, Max | 74.0, 83.0 | 69.0, 99.0 | 68.0, 110.0 | 68.0, 110.0 |
| Body Mass Index | N | 4 | 9 | 8 | 21 |
| kg/m² | Mean | 25.4 | 26.5 | 25.8 | 26.0 |
| | SD | 1.42 | 2.42 | 2.49 | 2.24 |
| | Median | 25.1 | 27.1 | 25.0 | 25.6 |
| | Min, Max | 24.2, 27.1 | 22.5, 29.7 | 23.6, 31.1 | 22.5, 31.1 |

### Measurement of Treatment Compliance

Subcutaneous dosing was administered by study site personnel.

### Pharmacokinetic Evaluations

Blood samples for PK analysis were drawn prior to dosing (within 30 minutes), and at 10, 20, and 45 minutes, and 1, 1.5, 2, 2.5, 3, 4, 6, 8, and 12 hours after dosing on Days 1 and 7. Additional PK sampling was done on Days 2, 3, 4, 5, and 7 (sample drawn prior to dosing) and on Days 8 and 11 at approximately 24 and 96 hours after the last dose. An additional PK sample was drawn at the Day 20 visit. These blood samples were not analyzed during the course of this study, but were collected into tubes containing peptidase inhibitors and processed to maintain stability of USB003.

### Evaluations for Pharmacologic Activity

Table 4 summarizes the shifts in CD3, CD34⁺, CD4, and CD8 cell subsets in peripheral blood from Baseline (Day 1) to Study Days 8 and Day 20 (Day 20±1).

Table 5 presents mean flow cytometry values at Baseline and mean changes from Baseline to Days 3, 5, 7, 8, 11, 14, 17, and 20 for CD3, CD34⁺, CD4, and CD8. The effects of USB003 on hematologic parameters are discussed further below.

TABLE 4
Flow Cytometry: Number of Subjects with Shifts in Mean CD3, CD34⁺, CD4, and
CD8 Cell Counts from Baseline to Study Days 8 and 20

| | | Treatment Group | | | | | | | | | | | |
|---|---|---|---|---|---|---|---|---|---|---|---|---|---|
| | | | | | | 200 μg/kg | | | | | | | |
| | | Placebo | | | | N = 8 for Day 8 | | | | 300 μg/kg | | | |
| | | N = 4 | | | | N = 9 for Day 20 | | | | N = 8 | | | |
| | Day | N-L | N-H | L-N | H-N | N-L | N-H | L-N | H-N | N-L | N-H | L-N | H-N |
| Absolute CD3 | 8 | — | — | — | — | — | — | — | 1 | — | — | — | — |
| | 20 | — | — | — | — | — | — | — | 1 | — | — | — | — |
| Absolute CD34 | 8 | — | — | — | — | — | — | — | — | — | — | — | — |
| | 20 | — | — | — | — | — | — | — | — | — | — | — | — |
| Absolute CD4 | 8 | — | — | — | — | — | — | — | 1 | — | — | 1 | — |
| | 20 | — | — | — | — | — | — | — | 1 | 1 | — | — | — |
| Absolute CD8 | 8 | — | — | — | — | — | 1 | — | — | — | 1 | — | — |
| | 20 | — | — | — | — | — | — | — | — | — | — | — | — |

Note:
N-L = normal to low;
N-H = normal to high;
L-N = low to normal;
H-N = high to normal;
—signifies subject with no shift from baseline.
Day 20 (200 μg/kg includes the Day 3 termination laboratory evaluations for Subject 011.

TABLE 5
Flow Cytometry: Mean Laboratory Values at Baseline and the Mean Change
from Baseline (Day 1), to Days 3, 5, 7, 8, 11, 14, 17, and 20

| | Treatment Group | | |
|---|---|---|---|
| Parameter Visit | Placebo | 200 μg/kg | 300 μg/kg |

FLOW CYTOMETRY
CD3, Absolute (cells/μL) (normal = 426-2767 cells/μL)

| | | | | | | | |
|---|---|---|---|---|---|---|---|
| Baseline | Mean (SD) | 1368.5 | (197.24) | 1732.3 | (545.85) | 1285.5 | (183.57) |
| Day 3 | Mean (SD) | −144.3 | (120.32) | −115.8 | (196.85) | 167.8 | (358.08) |
| Day 5 | Mean (SD) | −234.8 | (244.30) | 70.6 | (217.13) | 159.8 | (366.10) |
| Day 7 | Mean (SD) | −148.8 | (202.03) | 53.9 | (351.75) | 229.8 | (402.79) |
| Day 8 | Mean (SD) | −208.3 | (180.53) | −32.0 | (414.47) | 67.1 | (274.19) |
| Day 11 | Mean (SD) | −464.5 | (347.29) | 122.0 | (460.54) | −63.5 | (318.40) |
| Day 14 | Mean (SD) | −69.5 | (228.97) | 60.0 | (320.66) | 0.3 | (294.30) |
| Day 17 | Mean (SD) | −258.5 | (270.20) | −121.1 | (383.62) | −87.9 | (358.52) |
| Day 20 | Mean (SD) | −263.5 | (177.41) | −63.6 | (343.81) | 56.9 | (352.41) |

CD34⁺, Absolute (cells/μL) (normal = 0.7-6.9 cells/μL)

| | | | | | | | |
|---|---|---|---|---|---|---|---|
| Baseline | Mean (SD) | 4.13 | (1.563) | 2.50 | (1.286) | 2.55 | (1.114) |
| Day 3 | Mean (SD) | 0.43 | (1.584) | 0.23 | (0.539) | 1.80 | (1.625) |
| Day 5 | Mean (SD) | −1.08 | (1.274) | 0.61 | (1.328) | 0.56 | (1.126) |
| Day 7 | Mean (SD) | −0.70 | (0.726) | 1.11 | (1.216) | −0.01 | (1.270) |
| Day 8 | Mean (SD) | −0.63 | (0.885) | 0.33 | (0.997) | 0.88 | (1.075) |
| Day 11 | Mean (SD) | −2.13 | (1.438) | 0.13 | (1.012) | −0.74 | (0.873) |
| Day 14 | Mean (SD) | −1.60 | (1.003) | 0.61 | (1.524) | −0.41 | (0.590) |
| Day 17 | Mean (SD) | −1.03 | (0.918) | −0.40 | (0.421) | −0.31 | (1.260) |
| Day 20 | Mean (SD) | −1.33 | (1.100) | −0.46 | (1.000) | −0.35 | (1.088) |

CD4, Absolute (cells/μL) (normal = 508-1765 cells/μL)

| | | | | | | | |
|---|---|---|---|---|---|---|---|
| Baseline | Mean (SD) | 854.0 | (171.56) | 1122.6 | (335.42) | 734.4 | (148.80) |
| Day 3 | Mean (SD) | −132.5 | (181.73) | −88.0 | (128.64) | 109.6 | (237.13) |
| Day 5 | Mean (SD) | −165.8 | (245.10) | 39.5 | (155.09) | 108.1 | (265.03) |
| Day 7 | Mean (SD) | −122.5 | (178.93) | 53.6 | (230.20) | 129.3 | (232.22) |
| Day 8 | Mean (SD) | −162.0 | (183.35) | −24.0 | (246.95) | 31.1 | (164.06) |
| Day 11 | Mean (SD) | −319.5 | (143.45) | 82.5 | (280.71) | −29.3 | (211.29) |
| Day 14 | Mean (SD) | −68.0 | (190.94) | 12.1 | (193.73) | 9.7 | (195.91) |
| Day 17 | Mean (SD) | −199.8 | (226.82) | −70.5 | (248.63) | −40.0 | (367.05) |
| Day 20 | Mean (SD) | −190.0 | (151.51) | −58.0 | (200.27) | 48.9 | (227.23) |

CD8, Absolute (cells/μL) (normal = 216-1014 cells/μL)

| | | | | | | | |
|---|---|---|---|---|---|---|---|
| Baseline | Mean (SD) | 474.3 | (143.47) | 564.6 | (234.85) | 452.1 | (137.19) |
| Day 3 | Mean (SD) | −19.8 | (35.26) | −34.2 | (75.52) | 59.5 | (129.60) |
| Day 5 | Mean (SD) | −86.5 | (65.86) | 31.9 | (56.59) | 71.0 | (138.04) |
| Day 7 | Mean (SD) | −53.3 | (72.19) | 0.0 | (97.19) | 93.6 | (173.79) |
| Day 8 | Mean (SD) | −57.0 | (51.24) | −3.3 | (148.33) | 45.1 | (130.74) |
| Day 17 | Mean (SD) | −77.0 | (72.22) | −52.8 | (127.59) | −47.3 | (115.28) |
| Day 20 | Mean (SD) | −90.3 | (60.94) | −31.7 | (133.4) | 26.8 | (114.40) |

Several hematologic parameters were measured as part of this clinical study.

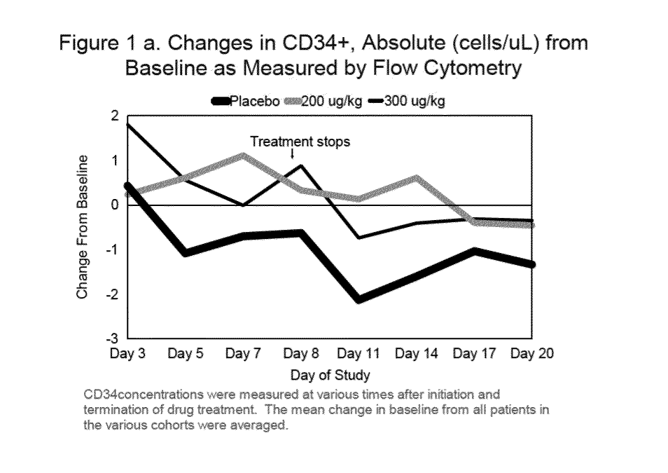

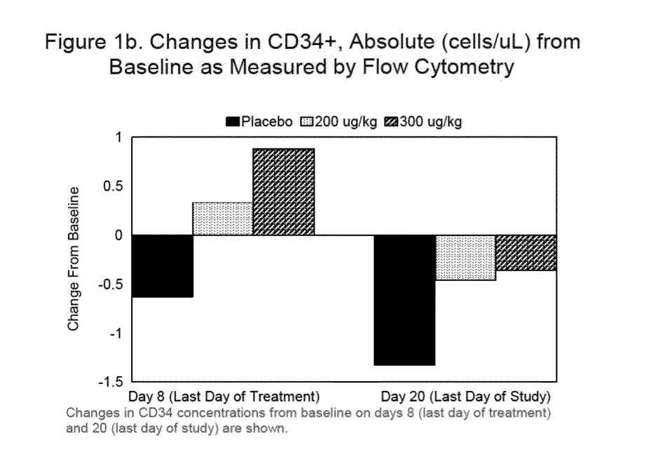

The data are presented as changes from baseline. There was a dose-related change in the number of CD34+ cells in the peripheral blood on the last day of treatment that was not observed 12 days after administration of the test article (FIG. 1). At the dose of 300 μg/kg/day, there was an approximately 15-20% increase in CD3 (pan T cell marker) during study drug administration that rapidly diminished after cessation of treatment (FIG. 2). A similar increase in CD4+ and CD8+ cells was observed during the treatment period (FIGS. 3 and 4).

### Safety Evaluation

### Extent of Exposure

Each subject received 1 SC dose of USB003 daily for 7 consecutive days. Subjects were to be dosed in 2 cohorts of 10. However, prior to randomization, several subjects in Cohort 1 withdrew consent. Seven subjects were randomized and dosed in the first cohort (Cohort 1a). After dosing on Day 3, one subject withdrew his consent. Four additional subjects were then dosed in Cohort 1b to complete the subjects required to be evaluated before escalating to the higher dose. All 10 subjects in Cohort 2 were dosed on the same day. Dosing groups and the date subjects received the first dose of study medication are presented in Table 6.

TABLE 6
Date of First Dose of Study Drug

| Day 1 | Subject Number | Dose Group |
|---|---|---|
| 30 Oct. 2002 | 003, 017 | Placebo |
| | 006, 007, 009, 011, 012 | USB003 (200 μg/kg) |
| 14 Nov. 2002 | 025, 027, 028, 033 | USB003 (200 μg/kg) |
| 05 Dec. 2002 | 41, 060 | Placebo |
| | 042, 043, 045, 046, 048, 052, | USB003 (300 μg/kg) |
| | 053, 055 | |

### Adverse Events

### Brief Summary of Adverse Events

There were no deaths or other serious adverse events (SAE), and no subject discontinued from the study because of AEs. Eleven of the 21 subjects (52.4%) experienced at least 1 AE. All but 1 AE had a CTC grade of 1 (0-4 scale). The other AE, a sprained ankle, was Grade 2 in severity. Upper respiratory infection, increased body temperature, increased ALT, increased WBC, and headache were each reported by more than 1 subject. Three subjects had 1 AE each (increased temperature, acid reflux, and upper respiratory infection) that was treated with a concomitant medication.

Five subjects (4 in the 300 μg/kg and 1 in placebo dose group) had elevations in their oral body temperature assessed as an AE. These were all assessed as Grade 1 toxicities and possibly related to study drug. Only 1 subject received treatment for this elevation in temperature, a single oral dose of 1 gram acetaminophen. The subjects were otherwise asymptomatic.

### Display of Adverse Events

All AEs were coded using the Medical Dictionary for Regulatory Activities Version 5.1 (MedDRA®).

### Analysis of Adverse Events

### All Adverse Events

The incidence of AEs was higher in the placebo group (75.0%) than in either the 200 μg/kg group (44.4%) or the 300 Ξg/kg group (50.0%). Table 7 presents the treat-emergent AEs reported in this study.

TABLE 7
Treatment-Emergent Adverse Events

| | | | USB003 | | USB003 | | | |
|---|---|---|---|---|---|---|---|---|
| | Placebo | | 200 μg/kg | | 300 μg/kg | | Overall | |
| Body System/Preferred term | (N = 4) | | (N = 9) | | (N = 8) | | (N = 21) | |
| MedDRA Terminology | Subjects | Events | Subjects | Events | Subjects | Events | Subjects | Events |
| Adverse Event n (%) | N (%) | (N) | N (%) | (N) | N (%) | (N) | N (%) | (N) |
| Subjects With At Least 1 AE | 3 (75.0) | 6 | 4 (44.4) | 7 | 4 (50.0) | 13 | 11 (52.4) | 26 |
| Gastrointestinal disorders | 1 (25.0) | 1 | 0 (0.0) | 0 | 1 (12.5) | 1 | 2 (9.5) | 2 |
| Gastroesophageal reflux | 1 (25.0) | 1 | 0 (0.0) | 0 | 0 (0.0) | 0 | 1 (4.8) | 1 |
| Nausea | 0 (0.0) | 0 | 0 (0.0) | 0 | 1 (12.5) | 1 | 1 (4.8) | 1 |
| Infections/Infestations | 1 (25.0) | 1 | 0 (0.0) | 0 | 1 (12.5) | 1 | 2 (9.5) | 2 |
| Upper respiratory tract | 1 (25.0) | 1 | 0 (0.0) | 0 | 1 (12.5) | 1 | 2 (9.5) | 2 |
| infections (NOS)¹ | | | | | | | | |
| Injury, poisoning and | 0 (0.0) | 0 | 0 (0.0) | 0 | 1 (12.5) | 1 | 1 (4.8) | 1 |
| procedural complications | | | | | | | | |
| Joint sprain | 0 (0.0) | 0 | 0 (0.0) | 0 | 1 (12.5) | 1 | 1 (4.8) | 1 |
| Investigations | 2 (50.0) | 2 | 2 (22.2) | 4 | 4 (50.0) | 9 | 8 (38.1) | 15 |
| Body temperature | 1 (25.0) | 1 | 0 (0.0) | 0 | 4 (50.0) | 7 | 5 (23.8) | 8 |
| increased | | | | | | | | |
| ALT increased | 0 (0.0) | 0 | 2 (22.2) | 2 | 0 (0.0) | 0 | 2 (9.5) | 2 |
| AST increased | 0 (0.0) | 0 | 1 (11.1) | 1 | 1 (12.5) | 1 | 2 (9.5) | 2 |
| WBC increased | 0 (0.0) | 0 | 1 (11.1) | 1 | 0 (0.0) | 0 | 1 (4.8) | 1 |
| Neutrophil count increased | 0 (0.0) | 0 | 0 (0.0) | 0 | 1 (12.5) | 1 | 1 (4.8) | 1 |
| White blood cells in urine | 1 (25.0) | 1 | 0 (0.0) | 0 | 0 (0.0) | 0 | 1 (4.8) | 1 |
| Neoplasms benign | 0 (0.0) | 0 | 1 (11.1) | 1 | 0 (0.0) | 0 | 1 (4.8) | 1 |
| Cyst NOS¹ | 0 (0.0) | 0 | 1 (11.1) | 1 | 0 (0.0) | 0 | 1 (4.8) | 1 |
| Nervous system disorders | 2 (50.0) | 2 | 0 (0.0) | 0 | 1 (12.5) | 0 | 3 (14.3) | 3 |
| Headache | 2 (50.0) | 2 | 0 (0.0) | 0 | 1 (12.5) | 0 | 3 (14.3) | 3 |
| Respiratory, thoracic and | 0 (0.0) | 0 | 1 (11.1) | 2 | 0 (0.0) | 0 | 1 (4.8) | 1 |
| mediastinal disorders | | | | | | | | |
| Nasal congestion | 0 (0.0) | 0 | 1 (11.1) | 1 | 0 (0.0) | 0 | 1 (4.8) | 1 |
| Pharyngitis | 0 (0.0) | 0 | 1 (11.1) | 1 | 0 (0.0) | 0 | 1 (4.8) | 1 |

¹Not otherwise specified

The body system with the highest incidence of AEs was Investigations with an incidence of 38.1% (15 events). The body system with the second highest incidence of AEs was Nervous system disorders with an incidence of 14.3% (3 events). The incidence of AEs in the remaining body systems were as follows: Gastrointestinal disorders and Infections/infestations 9.5% each; Injury, Neoplasms-benign, and Respiratory disorders 4.8% each.

When AEs are examined by dose group and preferred term for all subjects, it can be seen that incidence rates showed considerable variability across the 3 groups, probably because of the small sample size for each group. The only AEs that occurred in more than 1 subject and had a higher incidence in either USB003 group than in placebo was body temperature increase, which occurred in 50% of USB003 300 μg/kg vs 25% for placebo and ALT increase, which occurred in 22.2% of USB003 200 μg/kg vs 0.0% for placebo.

There were only 2 AEs that occurred in more than one subject in any USB003 treatment dose group. Increased body temperature occurred in 50% of the subjects at the 300 μg/kg dose versus 0.0% at the 200 μg/kg dose. ALT increase occurred in 22.2% of the subjects at the 200 μg/kg dose and 0.0% at the 300 μg/kg dose.

On inspection, all but one AE was a CTC Grade 1. Subject 046 (300 μg/kg) sustained an ankle sprain that was classified as Grade 2 severity on study Day 18. Three subjects were treated with concomitant medication for AEs. Subject 003 (Placebo) was treated with an oral antacid (2 tablespoons once a day) for acid reflux on Study Days 14 and 15. Subject 017 (Placebo) was treated with an over-the-counter cold and sinus preparation for 2 days starting on Study Day 11. Subject 042 (300 μg/kg) was given 1 dose of acetaminophen (1000 mg) for elevated temperature on Study Day 2.

For the 6 AEs reported by 3 subjects in the placebo group, the relationship to study drug was assessed as possible in 1, doubtful in 4, and not related in 1. Of the 7 events reported by 4 subjects in the 200 μg/kg group, 2 were assessed as possible, 3 doubtful, and 2 not related to study drug. Of the 13 events reported by 4 subjects in the 300 μg/kg group 8 were assessed as possible, 2 doubtful, and 3 not related to study drug.

Five subjects (4 in the 300 μg/kg, and 1 in placebo dose group) had elevations in their oral body temperature assessed as an AE and possibly related to study drug. The Investigator decided that any temperature >37.7° C. would be considered an AE.

### Adverse Events Related to Elevations in Oral Body Temperature.

Subject 042/CSW (300 μg/kg) was a 28-year-old Caucasian male who had a normal screening PE and was negative for any ongoing significant medical history. His body temperature at screen was 36.2° C. and at pre-dose on Day 1 it was 36.5° C. His temperature rose to 37.4° C. one hour after his first dose of study medication on Day 1. It was 37.8° C. at the 4-hour post-dose measurement and which was considered at this time to be an AE. It rose to 38.1° C. by the 9-hour post-dose time point and was 37.4° C. at the Day 2 pre-dose measurement. However by the 6-hour-post-dose time point on Day 2 it was 38.7° C. He was given 1 dose of 1000 mg oral acetaminophen. Between Days 3 and 6 his temperature fluctuated between 37.2° C. and 38.3° C. On Day 7 it ranged from 37.0° C. to 37.6° C. He left the clinic on Day 8 with a temperature of 36.9° C. No information on how the subject felt during these times of increase in body temperature is available. The Investigator assessed this increased body temperature as not serious, possibly related to the study drug, and considered resolved on Study Day 7. It was a CTC of Grade 1. This subject also experienced 2 other AEs which were recorded at the time of his Study Termination Visit on Day 19. His WBC value was 13,400 cells/mm³ and his segmented neutrophils were 84.9%. These AEs were considered resolved when a repeat test 10 days later showed the WBC count to be 8,900 cells/mm³ with 73.5% segmented neutrophils. The relationship of these AEs to study drug was assessed by the Investigator as doubtful.

Subject 046/CLC (300 μg/kg) was a 40-year-old Caucasian male who had a normal screening PE and was negative for any ongoing significant medical history. His body temperature at screen was 37.1° C. and at pre-dose on Day 1 it was 36.2° C. His temperature rose to 37.3° C. one hour after his first dose of study medication and continued to rise until it was 37.8° C., approximately 7 hours and 45 minutes after dosing and 38.0° C. at approximately 14 hours after the first dose. His temperature was a normal 35.9° C. at the pre-dose time point for Day 2. On Day 3 his temperature was 37.9° C. at the 6-hour post-dose time point, and was back to a normal 36.8° C. by the pre-dose measurement on Day 4. No information on how the subject felt during these times of increase in body temperature is available. The Investigator assessed these AEs as possibly related to study drug and resolved by the pre-dose vital signs on Day 4. They were a CTC of grade 1.

Subject 053/SJS (300 μg/kg) was a 22-year-old Caucasian male who had a normal screening PE and was negative for any ongoing significant medical history, except an allergy to aspirin. His body temperature at screen was 37.2° C., and at pre-dose on Day 1 it was 36.7° C. His temperature rose to 37.8° C. approximately 6 hours after his Day 3 study medication. It was a normal 36.4° C. by the Day 4 pre-dose vital sign measurements. His temperature rose to 37.9° C. approximately 6 hours after his Day 6 study medication. It was a normal 37.0° C. by the Day 7 pre-dose vital signs measurements. No treatment was given for this elevated temperature. No information on how the subject felt during these times of increase in body temperature is available. The Investigator assessed this temperature elevation as possibly related to study drug and a CTC of Grade 1.

Subject 055/PAW (300 μg/kg) was a 30-year-old Black male who had a normal screening PE and was negative for any ongoing significant medical history. His body temperature at screen was 36.2° C., and at pre-dose on Day 1 it was 36.3° C. His temperature rose to 38.0° C. approximately 6 hours after his Day 3 study medication. It was a normal 37.1° C. by the Day 4 pre-dose vital sign measurements. No medication was given. No information on how the subject felt during these times of increase in body temperature is available. The Investigator assessed this AE as possibly related to study medication. It was CTC of Grade 1.

Subject 060/RJH (Placebo) was a 36-year-old Caucasian male who had a normal screening PE and was negative for any ongoing significant medical history. His body temperature at screen was 36.8° C., and at pre-dose on Day 1 it was 36.9° C. His temperature rose to 37.9° C. approximately 6 hours after his Day 3 study medication. It was a normal 36.7° C. by the Day 4 pre-dose vital sign measurements. No medication was given. No information on how the subject felt during these times of increase in body temperature is available. The Investigator assessed this AE as possibly related to study medication. It was a CTC of Grade 1.

Adverse events, other than increased body temperature that were assessed as possibly related to study drug were transient ALT increase, transient skin cyst, and transient headache.

Subject 009/AOS (200 μg/kg) was a 25-year-old Caucasian male who had a normal screening PE and was negative for any ongoing significant medical history, except for seasonal allergies. At screen he had an ALT of 50 IU/L (Normal Range=0 to 50 IU/L) and an AST of 35 IU/L (Normal Range=10 to 40 IU/L). The pre-dose Day 1 values were high but not clinically significant (NCS) 54 IU/L for ALT, and a normal value of 34 IU/L for AST. The scheduled chemistry assessments on Day 8 showed an elevation of the ALT value to 103 IU/L, which was assessed as an AE. Though not assessed as an AE, the AST value had risen to a high but NCS at 66 IU/L. A repeat chemistry was performed on Day 14, and the ALT was high but NCS at 61 IU/L and the AST was a normal 29 IU/L. The Investigator assessed the AE of increased ALT as possibly related to study drug. It was a CTC score of Grade 1.

Subject 028 (200 μg/kg) was a 41-year-old Caucasian male who had poor dentition as a finding on his screening PE, and no ongoing significant medical history. On Day 7, he was found to have a cystic nodule on his anterior abdominal wall (presumably at an injection site). No other information about this was given. It was considered resolved without treatment on Study Day 10. The Investigator assessed this AE as possibly related to study drug. It was a CTC of Grade 1.

Subject 053/SJS (300 μg/kg) was a 22-year-old Caucasian male who had a normal screening PE and was negative for any ongoing significant medical history, except for allergy to aspirin. On Day 2 he experienced a headache 10 hours after the morning dose of study medication. It resolved without treatment 5 hours later. It was assessed by the Investigator as possibly related to study drug. It was a CTC of Grade 1.

### Evaluation of Each Laboratory Parameter

### Laboratory Values Over Time

No clinically important differences in the mean or median values between the 3 treatment groups could be seen for any analyte. Table 8 summarizes the median value for each of these analytes at baseline, Day 8 (end of treatment) and Day 20, and the median change in these values from baseline to Day 8 and Day 20. A relative increase in WBC concentration at 20 days was noted for the USB003 300 ug/kg group. In addition, an apparent dose-related increase in absolute neutrophil concentrations was noted at 20 days for the subjects that received 200 ug/kg and 300 ug/kg USB003.

TABLE 8
Summary of Median Laboratory Values at Baseline, Day 8, and Day 20 and Median Change
from Baseline to Days 8 and 20 for Selected Hematological Parameters

| | Treatment Group | | |
|---|---|---|---|
| Parameter Visit | Placebo (N = 4) | 200 μg/kg (N = 8) | 300 μg/kg (N = 8) |

HEMATOLOGY
Hemoglobin (normal range = 14-18 g/dL)

| | | | | | | | |
|---|---|---|---|---|---|---|---|
| Baseline | Median Min, Max | 13.75 | 13.0, 14.7 | 15.30 | 13.9, 17.2 | 15.60 | 12.7, 17.8 |
| Day 8 | Median Min, Max | 12.85 | 12.3, 15.8 | 15.10 | 13.9, 17.0 | 14.40 | 11.8, 15.9 |
| Day 20 | Median Min, Max | 13.15 | 12.3, 16.5 | 14.50 | 13.8, 16.4 | 14.0 | 12.1, 16.8 |
| Change from | Median Min, Max | −0.45 | −1.6, 1.1 | −0.20 | −0.9, 1.8 | −1.15 | −2.1, −0.7 |
| Baseline Day 8 | | | | | | | |
| Change from | Median Min, Max | −0.15 | −1.6, 1.8 | −0.70 | −1.6, 1.3 | −1.30 | −2.3, 0.4 |
| Baseline Day 20 | | | | | | | |

Hematocrit (normal range = 42-52%)

| | | | | | | | |
|---|---|---|---|---|---|---|---|
| Baseline | Median Min, Max | 41.25 | 40.0, 42.7 | 46.50 | 40.0, 52.0 | 45.25 | 39.2, 49.7 |
| Day 8 | Median Min, Max | 39.95 | 38.5, 46.6 | 43.65 | 39.7, 50.9 | 42.30 | 37.2, 45.3 |
| Day 20 | Median Min, Max | 39.45 | 38.1, 46.8 | 43.80 | 38.9, 48.4 | 40.45 | 37.0, 47.0 |
| Change from | Median Min, Max | −0.20 | −4.0, 4.2 | −1.65 | −4.0, −0.1 | −2.10 | −5.9, −0.8 |
| Baseline Day 8 | | | | | | | |
| Change from | Median Min, Max | −0.60 | −4.6, 4.4 | −1.50 | −5.1, 0.0 | −3.45 | −7.2, 0.8 |
| Baseline Day 20 | | | | | | | |

WBC (normal range = 4-11 thousands/mm³)

| | | | | | | | |
|---|---|---|---|---|---|---|---|
| Baseline | Median Min, Max | 6.45 | 5.2, 6.7 | 5.60 | 4.7, 10.7 | 5.15 | 4.0, 7.8 |
| Day 8 | Median Min, Max | 5.75 | 5.1, 6.2 | 6.65 | 5.3, 8.8 | 5.65 | 4.2, 7.2 |
| Day 20 | Median Min, Max | 5.40 | 4.6, 5.8 | 6.20 | 5.0, 9.2 | 6.55 | 4.2, 13.4 |
| Change from | Median Min, Max | −0.45 | −1.0, −0.1 | 0.35 | −2.6, 1.9 | −0.30 | −0.6, 2.1 |
| Baseline Day 8 | | | | | | | |
| Change from | Median Min, Max | −0.80 | −1.4, −0.6 | 0.40 | −3.7, 1.2 | 1.30 | −0.6, 5.6 |
| Baseline Day 20 | | | | | | | |

Absolute Neutrophils (normal range = 1.4-8.2 thousands/mm³)

| | | | | | | | |
|---|---|---|---|---|---|---|---|
| Baseline | Median Min, Max | 3.60 | 2.5, 4.4 | 3.00 | 2.3, 8.0 | 2.85 | 1.8, 5.7 |
| Day 8 | Median Min, Max | 3.40 | 2.5, 4.0 | 3.60 | 2.9, 5.6 | 2.95 | 1.9, 5.4 |
| Day 20 | Median Min, Max | 3.25 | 2.1, 3.4 | 3.80 | 2.3, 4.3 | 3.80 | 2.5, 11.3 |
| Change from | Median Min, Max | 0.05 | −1.3, 0.4 | 0.45 | −3.1, 1.1 | −0.10 | −0.7, 1.5 |
| Baseline Day 8 | | | | | | | |
| Change from | Median Min, Max | −0.55 | −1.0, 0.0 | 0.20 | −3.7, 1.2 | 0.75 | −0.8, 5.6 |
| Baseline Day 20 | | | | | | | |

Lymphocytes (normal range = 24-44%)

| | | | | | | | |
|---|---|---|---|---|---|---|---|
| Baseline | Median Min, Max | 36.30 | 23.9, 39.6 | 35.80 | 17.8, 46.0 | 33.60 | 18.3, 45.5 |
| Day 8 | Median Min, Max | 33.15 | 24.7, 34.7 | 32.40 | 27.1, 40.9 | 34.50 | 18.5, 47.8 |
| Day 20 | Median Min, Max | 30.90 | 26.4, 34.0 | 31.50 | 30.0, 48.6 | 29.75 | 10.1, 40.9 |
| Change from | Median Min, Max | −5.80 | −9.1, 10.3 | −2.25 | −8.7, 12.5 | 1.00 | −3.8, 6.9 |
| Baseline Day 8 | | | | | | | |
| Change from | Median Min, Max | −5.30 | −5.8, 2.5 | −1.80 | −10.1, 13.0 | −3.90 | −10.1, 6.1 |
| Baseline Day 20 | | | | | | | |

Platelet Count (normal range = 150-400 thousands/mm³)

| | | | | | | | |
|---|---|---|---|---|---|---|---|
| Baseline | Median Min, Max | 284.0 | 218, 339 | 263.0 | 205, 377 | 216.0 | 134, 301 |
| Day 8 | Median Min, Max | 228.5 | 142, 336 | 242.0 | 203, 412 | 243.0 | 192, 301 |
| Day 20 | Median Min, Max | 262.5 | 146, 309 | 250.0 | 211, 484 | 243.5 | 173, 329 |
| Change from | Median Min, Max | −71.5 | −91, 44 | 1.0 | −24, 35 | 22.5 | −25, 113 |
| Baseline Day 8 | | | | | | | |
| Change from | Median Min, Max | −30.5 | −72, −12 | −5 | −62, 107 | 14 | −40, 115 |
| Baseline Day 20 | | | | | | | |

There were no notable effects of USB003 on the mean change from baseline to Day 4 or Day 8 on any serum chemistry evaluations.

The effects of USB003 on kidney function are summarized in Table 9.

TABLE 9
Summary of Renal Laboratory Values at Baseline and Change in Laboratory Values from
Baseline (Day 1) to Day 8

| | Treatment Group | | |
|---|---|---|---|
| Parameter Visit | Placebo | 200 μg/kg | 300 μg/kg |

Serum Creatinine (mg/dL)

| | | | | | | | |
|---|---|---|---|---|---|---|---|
| Baseline | Mean (SD) | 1.00 | (0.115) | 1.08 | (0.254) | 1.06 | (0.130) |
| | Median | 1.00 | | 1.00 | | 1.00 | |
| | Min, Max | 0.9 | 1.1 | 0.9 | 1.7 | 0.9 | 1.3 |
| Day 8 | Mean (SD) | 1.05 | (0.100) | 1.03 | (0.139) | 1.10 | (0.160) |
| | Median | 1.00 | | 1.00 | | 1.05 | |
| | Min, Max | 1.0 | 1.2 | 0.9 | 1.3 | 0.9 | 1.4 |
| Change from | Mean (SD) | 0.05 | (0.100) | −0.06 | (0.141) | 0.04 | (0.052) |
| Baseline | Median | 0.10 | | 0.00 | | 0.00 | |
| | Min, Max | −0.1 | 0.1 | −0.4 | 0.0 | 0.0 | 0.1 |

Urine
Creatinine Clearance (mL/min/1.73 m³)

| | | | | | | | |
|---|---|---|---|---|---|---|---|
| Baseline | Mean (SD) | 102.75 | (27.837) | 98.67 | (21.237) | 103.38 | (15.874) |
| | Median | 100.00 | | 101.00 | | 99.50 | |
| | Min, Max | 72.0 | 139.0 | 58.0 | 125.0 | 85.0 | 135.0 |
| Day 8 | Mean (SD) | 105.75 | (21.376) | 119.38 | (10.809) | 104.00 | (21.791) |
| | Median | 108.00 | | 120.50 | | 101.50 | |
| | Min, Max | 78.0 | 129.0 | 102.0 | 131.0 | 75.0 | 144.0 |
| Change from | Mean (SD) | 3.00 | (26.064) | 24.00 | (12.271) | 0.63 | (22.557) |
| Baseline | Median | −6.00 | | 24.00 | | −3.50 | |
| | Min, Max | −17.0 | 41.0 | 5.0 | 44.0 | −27.0 | 38.0 |

Creatinine, Urine (normal = 1-2 g/24 h)

| | | | | | | | |
|---|---|---|---|---|---|---|---|
| Baseline | Mean (SD) | 1.75 | (0.311) | 1.76 | (0.573) | 1.98 | (0.341) |
| | Median | 1.75 | | 1.90 | | 2.00 | |
| | Min, Max | 1.4 | 2.1 | 1.0 | 2.8 | 1.5 | 2.5 |
| Day 8 | Mean (SD) | 1.83 | (0.435) | 2.03 | (0.459) | 1.90 | (0.424) |
| | Median | 1.95 | | 1.95 | | 1.95 | |
| | Min, Max | 1.2 | 2.2 | 1.5 | 2.9 | 1.2 | 2.5 |
| Change from | Mean (SD) | 0.08 | (0.369) | 0.33 | (0.238) | −0.08 | (0.328) |
| Baseline | Median | 0.10 | | 0.25 | | −0.15 | |
| | Min, Max | −0.4 | 0.5 | 0.1 | 0.8 | −0.4 | 0.5 |

Potassium, 24-Hour (normal = 26-123 mEq/24 h)

| | | | | | | | |
|---|---|---|---|---|---|---|---|
| Baseline | Mean (SD) | 68.50 | (19.672) | 59.89 | (19.592) | 52.25 | (17.766) |
| | Median | 68.0 | | 52.00 | | 51.00 | |
| | Min, Max | 51.0 | 87.0 | 41.0 | 93.0 | 32.0 | 86.0 |
| Day 8 | Mean (SD) | 59.75 | (19.346) | 63.00 | (14.333) | 59.63 | (11.673) |
| | Median | 67.50 | | 64.50 | | 60.50 | |
| | Min, Max | 31.0 | 73.0 | 42.0 | 78.0 | 36.0 | 74.0 |
| Change from | Mean (SD) | −8.75 | (19.873) | 4.63 | (21.246) | 7.38 | (19.632) |
| Baseline | Median | −18.00 | | 8.5 | | 11.0 | |
| | Min, Max | −20.0 | 21.0 | −30.0 | 35.0 | −30.0 | 36.0 |

Sodium, 24-Hour Urine (normal = 80-210 mEq/24 h)

| | | | | | | | |
|---|---|---|---|---|---|---|---|
| Baseline | Mean (SD) | 202.25 | (47.549) | 184.67 | (55.978) | 178.13 | (50.843) |
| | Median | 187.0 | | 180.00 | | 199.50 | |
| | Min, Max | 164.0 | 271.0 | 81.0 | 279.0 | 93.0 | 238.0 |
| Day 8 | Mean (SD) | 191.25 | (26.600) | 209.38 | (22.148) | 208.63 | (47.770) |
| | Median | 193.50 | | 216.50 | | 227.50 | |
| | Min, Max | 158.0 | 220.0 | 169.0 | 237.0 | 126.0 | 254.0 |
| Change from | Mean (SD) | −11.00 | (37.417) | 36.50 | (55.188) | 30.50 | (67.212) |
| Baseline | Median | −16.00 | | 29.50 | | 34.50 | |
| | Min, Max | −51.0 | 39.0 | −28.0 | 139.0 | −88.0 | 129.0 |

The increase in creatinine clearance observed at the 200 μg/kg dose level did not correlate with a decrease in serum creatinine, and was not observed in the 300 μg/kg dose level. Slight increases in 24-hour urinary excretion of sodium were also observed in both treatment groups.

### Individual Subject Change

Table 9 summarizes shifts in hematology, blood chemistry, and urine laboratory parameters from Day 1 to Day 8 and Day 20. In general, there were few shifts from normal to out-of-range, for either hematology or chemistry parameters, except for Hb and Hct which is consistent with studies having multiple blood draws. Compared with placebo, a notable number of subjects receiving USB003 had shifts in eosinophils from high at baseline to normal at Day 8 and Day 20 in the 200 μg/kg dose group. Consistent with the findings in Section 12.4.2.1 is the finding that the creatinine clearance shifted from low to normal in 3 of the 9 subjects in the 200 μg/kg group, and in 2 of the 8 subjects in the 300 μg/kg group. The urinary sodium excretion went from normal to high in 4 of 9 and 5 of 8 subjects in the 200 and 300 μg/kg dose groups, respectively.

TABLE 9
Summary of Laboratory Value Shifts from Baseline to Out-of-Range or to Normal on
Protocol Specified Dates

| | Number of Subjects According to Treatment Group | | | | | | | | | | | |
|---|---|---|---|---|---|---|---|---|---|---|---|---|
| | Placebo | | | | 200 μg/kg | | | | 300 μg/kg | | | |
| | N = 4 | | | | N = 9 | | | | N = 8 | | | |
| Day | N-L | N-H | L-N | H-N | N-L | N-H | L-N | H-N | N-L | N-H | L-N | H-N |

Hematology

| | | | | | | | | | | | | | |
|---|---|---|---|---|---|---|---|---|---|---|---|---|---|
| Absolute Eosinophils | 8 | — | — | — | — | — | — | — | 1 | — | — | — | — |
| | 20 | — | — | — | — | — | 1 | — | — | — | — | — | — |
| Absolute Neutrophils | 20 | — | — | — | — | — | — | — | — | — | 1 | — | — |
| Eosinophils | 8 | — | 1 | — | — | — | — | — | 3 | — | — | — | 1 |
| | 20 | — | 1 | — | — | — | — | — | 2 | — | 2 | — | 1 |
| Hematocrit | 8 | 1 | — | — | — | — | — | — | — | 2 | — | — | — |
| | 20 | 1 | — | — | — | — | — | — | — | 4 | — | — | — |
| Hemoglobin | 8 | — | — | — | — | — | — | — | — | 2 | — | — | — |
| | 20 | — | — | — | — | 1 | — | 1 | — | 3 | — | — | — |
| Lymphocytes | 8 | — | — | 1 | | — | — | 1 | 1 | — | — | — | — |
| | 20 | — | — | 1 | — | — | — | 1 | — | — | — | — | 1 |
| Monocytes | 20 | — | 1 | — | — | — | — | — | — | — | — | — | — |
| Plasma Hemoglobin | 8 | — | 1 | — | — | — | — | — | — | — | — | — | — |
| | 20 | — | — | — | — | — | 2 | — | — | — | 1 | — | — |
| Platelet Count | 8 | 1 | — | — | — | — | 1 | — | — | — | — | 1 | — |
| | 20 | 1 | — | — | — | — | 1 | — | — | — | — | 1 | — |
| RBC | 8 | 2 | — | — | — | — | — | — | — | 1 | — | — | — |
| | 20 | 1 | — | — | — | — | — | — | — | 2 | — | — | — |
| RDW | 20 | — | — | — | — | — | 1 | — | — | — | — | — | — |
| Absolute Reticulocytes | 8 | — | 1 | — | — | — | — | — | — | — | 1 | — | — |
| | 20 | — | — | — | — | — | 2 | — | — | — | — | — | — |
| Segmented Neutrophils | 8 | — | 1 | — | — | — | — | — | 1 | — | — | — | — |
| | 20 | — | — | — | — | — | — | — | 1 | — | 2 | — | — |
| WBC | 20 | — | — | — | — | — | — | — | — | — | 1 | — | — |

Chemistry

| | | | | | | | | | | | | | |
|---|---|---|---|---|---|---|---|---|---|---|---|---|---|
| ALT (SGPT) | 8 | — | 1 | — | — | — | 1 | — | — | — | — | — | — |
| AST (SGOT) | 8 | — | 1 | — | — | — | 1 | — | — | — | — | — | — |
| BUN | 8 | — | — | 1 | — | — | — | 1 | — | — | — | 1 | — |
| Creatinine | 8 | — | — | — | — | — | — | — | 1 | — | — | — | — |
| Magnesium | 8 | — | — | — | — | 1 | — | — | — | — | — | 1 | — |
| Potassium | 8 | 1 | — | — | — | — | — | — | — | 1 | — | — | 1 |
| Total CPK | 8 | — | — | — | — | — | — | — | 1 | — | — | — | — |

Urinalysis

| | | | | | | | | | | | | | |
|---|---|---|---|---|---|---|---|---|---|---|---|---|---|
| Creatinine Clearance | 8 | — | — | — | — | — | — | 3 | — | 1 | — | 2 | — |
| Creatinine Urine | 8 | — | — | — | — | — | 3 | — | — | — | 1 | — | 2 |
| Potassium, 24 Hour | 8 | — | — | — | — | — | — | — | — | — | — | — | — |
| Sodium, 24 Hours | 8 | — | — | — | — | — | 4 | — | — | — | 5 | — | 2 |

Note:
N = normal;
H = High;
L = Low;
CPK = creatine phosphokinase;
RDW = red cell distribution width.
“—” = 0.
Baseline is defined as the last measurable value prior to dosing on Day 1.

### Individual Clinically Significant Abnormalities

Five subjects had at least 1 clinically significant (CS) out-of-range value at some time after the Screening Visit (Reference: individual laboratory reports in CRFs). All of these CS abnormalities were assessed as AEs. The greatest number of CS values was observed in the 200 μg/kg dose group (3 subjects had a total of 6 CS abnormalities). This was followed by the 300 μg/kg dose group (1 subject had 2 CS abnormalities), and then placebo (1 subject had 1 CS abnormality). Table 10 lists laboratory values that were assessed as CS, as well as follow-up (and when appropriate, previous) laboratory values, until the analyte was assessed as normal or NCS. The Investigator wrote the clinical significance of all out-of range laboratory values directly on the laboratory report. However, this was not transcribed to an actual page on the CRF. Hence, these laboratory reports with the Investigator's assessment can only be found written on the laboratory results in the CRF.

TABLE 10
Summary of Laboratory Values that were Assessed as Clinically Significant and an Adverse
Event with Follow-up Test Results (Intent-to-Treat)

| | Dose | | | | | High/Low | |
|---|---|---|---|---|---|---|---|
| Subject | Group | Day | Date | Analyte | Value | Normal/Abnormal | Significance |
| 003 | Placebo | −8 | 22 Oct 2002 | Urine WBC | 12 | H | CS |
| | | −1 | 29 Oct 2002 | | 0 | N | |
| | | 8 | 06 Nov 2002 | | 12 | H | CS AE |
| | | 14 U | 12 Nov 2002 | | 0 | N | |
| 009 | 200 μg/kg | −8 | 22 Oct 2002 | ALT/SGPT | 50 | N | |
| | | −1 | 29 Oct 2002 | | 57 | H | NCS |
| | | 1 | 30 Oct 2002 | | 54 | H | NCS |
| | | 4 | 02 Nov 2002 | | 59 | H | NCS |
| | | 8 | 06 Nov 2002 | | 103 | H | CS AE |
| | | 14 U | 12 Nov 2002 | | 61 | H | NCS |
| 011 | 200 μg/kg | −8 | 22 Oct 2002 | Urine Glucose | Trace | Abnormal | CS |
| | | −1 | 29 Oct 2002 | | Neg | N | |
| | | 1 | 30 Oct 2002 | | +1 | Abnormal | CS AE |
| | | 3 | 01 Nov 2002 | | neg | N | |
| 027 | 200 μg/kg | 4 | 17 Nov 2002 | AST/SGOT | 72 | H | CS AE |
| | | 7 U | 20 Nov 2002 | | 25 | N | |
| | | 8 | 21 Nov 2002 | | 24 | N | |
| | | 4 | 17 Nov 2002 | ALT/SGPT | 93 | H | CS AE |
| | | 7 U | 20 Nov 2002 | | 68 | H | NCS |
| | | 8 | 21 Nov 2002 | | 61 | H | NCS |
| | | −1 | 13 Nov 2002 | WBC | 11.4 | H | NCS |
| | | 11 | 24 Nov 2002 | | 12.7 | H | CS AE |
| | | 14 | 27 Nov 2002 | | 7.3 | N | |
| 042 | 300 μg/kg | 19 | 23 Dec 2002 | WBC | 13.4 | H | CS AE |
| | | 29 U | 02 Jan 2003 | | 8.9 | N | |
| | | 19 | 23 Dec 2002 | Segmented Neutrophils | 84.9 | H | CS AE |
| | | 29 U | 02 Jan 2003 | | 73.5 | H | NCS |

Note:
H = High;
N = Normal;
CS = Clinically Significant;
NCS = Not Clinically Significant;
AE = Adverse event;
U = unscheduled

All high or abnormal laboratory values that were assessed as AEs were followed until resolution. The relationship of all these CS AEs to USB003 was assessed as doubtful in all cases, except the serum ALT/SGPT of Subject 009 on Day 8 that was assessed as possibly related to study drug.

### Vital Signs, Physical Findings, and Other Observations Related to Safety

### Vital Signs

Vital signs, including SBP, DBP, RR, and HR were done at Screening, on each day immediately before and at 1, 3, and 6 hours after each SC injection, and at 24 hours after the last injection on Day 7. On Day 1, they were taken every hour for the first 6 hours, then every 3 hours up to 12 hours after the dose.

### Pulse Rate

In general, on most days, there seemed to be a trend for a mildly notable increase in the mean change in pulse rate by the 3-hour post-dose time point in all treatment groups, and continuing in the USB003 groups at the last measured vital sign of the day (12-hour on Day 1 and 6-hours post-dose on other days).

### Systolic and Diastolic Blood Pressures

In general, there was a mild decrease in both the change in mean SBP and DBP, as compared to baseline for many measured time points. However there was no apparent difference in dose groups and no trend in time after dosing could be clearly seen.

### Oral Body Temperature

There was a clearly notable increase in the change from baseline in mean body temperatures for the 300 μg/kg dose group, compared to the 200 μg/kg dose. This was especially evident at the 6-hour post-dose time point, and although it occurred at other times, it was seen most frequently on Study Day 3. One subject in the placebo group had a mildly notable change in the mean oral body temperature, but this was only on the third day of dosing.

### Physical Findings

For all dose groups, there were no changes from baseline (Day 1) to Day 20 Termination Visit for any PE finding, except that it was noted on the discharge (Day 20) PE of Subject 46 (who had the AE of a sprained ankle during the study) that there was a “posterior splint to right ankle/right lower leg secondary to wrestling injury . . . ”.

### Respiration Rate

There were no clinically meaningful changes from baseline to all measured time points for the mean RR.

### Electrocardiograms

There were no abnormal CS 12-lead ECGs findings at Screening or on Study Day 8.

### Safety Conclusions

In general, USB003, at SC doses of 200 and 300 μg/kg was safe and well tolerated.
There were no deaths or SAEs, and no subject discontinued from the study because of an AE.
Five subjects (4 in the 300 μg/kg and 1 in placebo dose group) did have elevations in their oral body temperature high enough to be assessed as an AE. These were all assessed as possibly related to study drug. They all had a CTC score of Grade 1, but were otherwise asymptomatic.
Adverse events, other than increased temperature, that were assessed as possibly related to study drug were increased ALT, headache, and cystic nodule on anterior abdominal wall.
Adverse events that occurred during the study, whose relationship to study drug was assessed as doubtful, included increased ALT and AST, headache, acid reflux, WBCs in the urine and glucose in urine.
Upper respiratory infection, increased body temperature, increased ALT, increased WBC, and headache were each reported in more than 1 subject.
There were no AEs with CTC scores of Grade 3 or higher and only 1 Grade 2 toxicity.
Three subjects had 1 AE each that required treatment with concomitant medication (increased temperature, acid reflux, and upper respiratory infection).
All AEs resolved by the Study Termination (except ankle sprain), and there were no changes in clinical laboratory tests, 12-lead ECGs, vital signs, or PEs that were of continuing clinical concern.
There was an increase in creatinine clearance observed at the 200 μg/kg dose level but it did not correlate with a decrease in serum creatinine, and was not observed in the 300 μg/kg dose level.
A slight increase in the 24-hour urinary excretion of sodium was observed in both treatment groups.

### Discussion and Overall Conclusions

USB003 was safe and well tolerated at both the 200 and 300 μg/kg SC doses. There were no SAEs and no AE led to withdrawal from the study. Increased body temperature, increased WBC, and isolated liver enzyme elevations were the only AEs that occurred in more than one subject taking USB003.
USB003 had an effect on hematologic parameters. The findings were increases in total WBC and neutrophil concentrations. In addition, concentrations of CD3, CD34+, CD4 and CD8 were increased during USB003 administration then recovered to baseline levels within 12 days following discontinuation of therapy.
At the dose of 300 μg/kg/day (but not at 200 ug/kg/day), there was an approximately 15-20% increase in CD3 (pan T cell marker) during study drug administration that rapidly diminished after cessation of treatment (FIG. 2). A similar increase in CD4+ and CD8+ cells was observed during the treatment period (FIGS. 3 and 4).

### Example 2

### A Phase I Evaluation of the Safety and Biologic Activity of TXA127 in HIV-Infected Subjects with CD4+ T-Lymphocyte Counts Less Than 200 per mm³ Who Have Responded to HAART

### Introduction

In the absence of effective antiretroviral therapy, HIV disease is characterized by the relentless destruction of CD4+ T-lymphocytes resulting in disordered and declining immune function. TXA127 [Angiotensin 1-7 A(1-7), formulated for administration to humans] is a lymphocyte growth factor and in human subjects without HIV infection (e.g., breast cancer subjects receiving cancer chemotherapy) dramatic recovery of lymphocyte numbers were observed following the subcutaneous injection of TXA127. This Phase I dose escalation clinical study will evaluate the safety and biologic activity of TXA127 in a selected population of HIV seropositive individuals whose serum HIV RNA viral loads have become undetectable with highly active antiretroviral therapy (HAART).

### Protocol Synopsis

### Disease Immune Reconstitution in Human Immunodeficiency Virus Infection

### Drug Category: Lymphocyte Stimulating Factor

### Objectives:

### Primary:

To assess the safety and determine the maximum tolerated dose (MTD) of A(1-7) (referred to also herein as “TXA127”) administered by subcutaneous injection in HIV-infected subjects with CD4+ T-lymphocyte counts less than 200 per mm³ who have responded to highly active antiretroviral therapy (HAART).

### Secondary:

1. To assess the frequency of subjects achieving a target CD4+ T-lymphocyte count >350 per mm³.
2. To assess the durability of response once TXA127 is stopped.

### Study Design:

This is a Phase I, single institution, open-label, within-dosing-cohort-schedule randomized, dose escalation study of TXA127 in HIV-infected subjects with CD4+ T-lymphocyte counts less than 200 per mm³ who have responded to HAART. The study has been designed to determine the MTD of TXA127 in this subject population. This study will also obtain safety and biologic activity information about the subcutaneous injection of TXA127.

A standard Simon Phase I dose escalation trial has been proposed. The MTD will have been exceeded if the proportion of subjects that develops the same or similar study-drug-related, dose-limiting toxicity (DLT) in an assigned dosing schedule equals 2/2, 2/3, 2/4, 2/5, and 2/6 subjects. The MTD is defined as the largest dose that ≦1 of 6 subjects experiences a DLT. Dose-limiting toxicity is defined as a drug-related grade 3 or 4 adverse event (AE).

### Study Subjects:

### Inclusion Criteria:

HIV-infected males or non-pregnant, non-breast feeding females who are ≧18 years of age; CD4+ T-lymphocyte count less than 200 per mm³; successful response to HAART (HIV RNA viral load of <400 copies per mL) for a minimum of 12 weeks preceding study enrollment and with an HIV RNA viral load of <50 copies/mL at study screening.

### Exclusion Criteria:

A history of an opportunistic infection within the 6 months prior to study enrollment; a history of active tuberculosis or other mycobacterial infection; uncontrolled high blood pressure (systolic blood pressure >180 mmHg or diastolic blood press >90 mmHg); congestive heart failure class III or IV; use of systemic glucocorticoid therapy or any other immunomodulators within 30 days of study entry including granulocyte colony stimulating factor (G-CSF), granulocyte macrophage colony stimulating factor (GM-CSF), interleukin 2 (IL-2), interferon, and erythropoietin; ACE inhibitors; Peroxisome Proliferator-Activated Receptor (PPAR) Gamma agonists; angiotensin II antagonists; a prior history of Kaposi's sarcoma; a prior history of lymphoma; women who are breast feeding; active substance abuse (cocaine, heroin, amphetamines, or alcohol at >30 mL/day) within the last 30 days; subjects with uncontrolled psychiatric disorders, including depression; aspartate aminotransferase (AST) or alanine aminotransferase (ALT)>3.0× upper limit of normal (ULN); serum creatinine >2.5×ULN; hemoglobin <10.0 g/dL; absolute neutrophil count (ANC)<750/mm³; platelet count <75,000/mm³; the use of an investigational drug in the previous 30 days; and any other condition that, in the opinion of the Investigator, may interfere with the subject's ability to comply with the study requirements or visit schedule.

### Enrollment and Dosing

Four escalating dosing cohorts will be examined to determine the MTD. The four dosing cohorts will receive 50, 100, 200 and 300 μg/kg of TXA127 by subcutaneous injection daily for 14 days then 14 days without treatment. The 28 days will be defined as one cycle. The cycle of therapy will be repeated once, for a total of two courses of treatment. Dose escalation to the next cohort of subjects will be permitted to the next higher dosing level provided the following criteria have been met.

The Protocol Chair and the Safety Monitor will confer twice monthly to review the status of the previous subjects and determine how to proceed by applying the following rules to determine whether the MTD has been exceeded or if additional subjects or dosing cohorts should be examined:
1. The initial three subjects in each dosing cohort will be enrolled one at a time, at intervals of no less than 14-days, receive their assigned dose of TXA127, and observed for development of DLTs.
2. If, after a minimum of 14 days following the initiation of injections by the third subject in a given dose cohort, no DLT and no increase in HIV viral loads above 3000 copies/mL has occurred in any of the initial three subjects, then the dose will be escalated to the next higher dose level, and new subjects enrolled into the next cohort.
3. If, after a minimum of 14 days following the initiation of injections by the third subject in a given dose cohort, any one subject out of the initial three develops a DLT, then up to three more subjects will be added to that dose cohort.
4. If, after a minimum of 14 days following the initiation of injections by the sixth subject in a given dose cohort, no additional subjects in the second set of three subjects develop a DLT (in other words, only one of six subjects at a given dose level develops a DLT), then the dose will be escalated to the next higher dose level, and new subjects enrolled into the next cohort.
5. If a second subject at a specific dose level develops the same or similar DLT, even if it is before there are six total subjects in that dose cohort, then the MTD will have been exceeded and no additional subjects will be added to this or any higher dose levels. [Note that if this occurs within the initial dosing cohort (50 μg/kg), the study will TERMINATE.]
6. If the proportion of subjects at a given dose level developing a DLT exceeds one of 3 subjects or one of 6 subjects, then the MTD will have been exceeded and the next lower dose will be considered the MTD.
7. If the 300 μg/kg dose is reached without having determined the MTD, then subjects will continue to be enrolled at this dose level following the same logic as expressed in the steps above, except that there will be no further escalation past 300 μg/kg.
8. Once the dose that appears to be the MTD is determined, additional subjects will be added to that dose cohort, one at a time, at intervals of no less than 14 days, until the total number of subjects at the maximum tolerated dose level equals nine, and no more than two of them have developed the same or similar DLT after at least 14 days since the last subject initiated their injections.
   Acceptable proportions of subjects developing a DLT to subjects examined at a specific dose level are zero, 1/3, 1/4, 1/5, and 1/6; and at the dose level determined to be the MTD, 2/7, 2/8 and 2/9. Proportions exceeding the MTD rule and requiring returning to the next lower dose (or terminating, if the dose is 50 μg/kg) include 2/2, 2/3, 2/4, 2/5, and 2/6.

### Sample Size:

The minimum number of evaluable subjects in this study will be two (two on the dosing schedule of 50 μg/kg). The maximum number will be 36 if the dose escalates to the highest level and is then de-escalated to the lowest level. The anticipated number of evaluable subjects enrolled will be 18 (three in each of the 3 lower cohorts and nine at the 300 μg/kg dose level); however, replacements may be needed, e.g., if a subject fails to have any of the first three follow-up visits.

### Data Analysis:

Toxicity data will be presented by severity level for each cohort dose level. The incidence of adverse events and treatment discontinuations will be summarized for each dose group. It is anticipated that Fisher's Exact test will be used to compare the dose groups with respect to the incidence of specific adverse events. The following analyses will be utilized to explore on a preliminary basis the possibility of efficacy of TXA127: For each dose group, and for all groups combined, 95% confidence intervals will be constructed to evaluate the proportion of subjects that increase their CD4+ T-lymphocyte counts. No hypothesis testing will take place. Serial measurements of CD4+ T-lymphocyte counts will be used for determination of biologic activity.

### Statistical Considerations

### General Design

This is a Phase I, single institution, open-label, cohort dose escalation study to assess the safety of TXA127 in subjects with HIV who have less than 200 CD4+ T-lymphocyte counts who have clinically and virologically responded to HAART. Four treatment cohorts are planned for study. Cohorts will consist of subjects entered at the same dose level. The cohorts will accrue at most 3 additional subjects at each dose schedule if one of the first 3 subjects experiences a DLT within 14 days of administration of study material. Subjects will be followed for a total of 6 months. At enrollment, subjects will be assigned to one of the four cohorts after the Protocol Team has fully reviewed each previous cohort and given its approval to proceed to the next cohort.

The primary objective of this study is to assess the safety and determine the maximum tolerated dose (MTD) of TXA127 administered by subcutaneous injection in HIV-infected subjects who have responded to HAART.

The maximum tolerated dose (MTD) is defined as: the dose of the cohort immediately preceding the cohort with greater than or equal to one-third of the subjects experiencing a DLT. A DLT is defined as a grade 3 or 4 adverse event which is possibly, probably, or definitely study treatment related. If DLTs occur in greater than or equal to one-third of the subjects at a particular dose level and dosing schedule, then the dose level will exceed the maximum tolerated dose and no additional subjects will be added to this or higher doses at the same dosing schedule. Other objectives will be to determine biological effect and to conduct studies of the ability of TXA127 to repopulate the CD4+ T-lymphocyte population of study subjects.

### Evaluation of Safety

An intent to treat (ITT) analysis will be performed on all safety data. That is, all subjects that receive at least one dose of study medication will be included in the analysis of safety and tolerability. All data will be summarized within the cohorts as well as between the treatment arms. No inferential statistics will be performed unless shift analysis indicates a clinically meaningful pre- to post-difference in the frequency of an adverse event or laboratory perturbation.

### Maximum Tolerated Dose

The primary objective of this study will be to determine the MTD of TXA127. Dose cohort will provide a listing of all DLTs by dose group and subject along with a summary table giving the number of DLTs.

### Effect on Karnofsky Performance Status

The serial measurements of Karnofsky Performance Status will be evaluated longitudinally as change from baseline and presented in summary form within each dosage cohorts, between the cohorts, and between the treatment arms.

### Viral Load

Viral load data will be compared between baseline, and day 14 of each treatment cycle and monthly following the second treatment cycle in order to determine if TXA127 affects viral replication. Paired t-tests on the logarithmic transformed viral load data will be utilized on all subjects with paired data.

### Adverse Events

The incidence of AEs will be presented by frequency, body system, severity, and relationship to study medication. In addition, the associated toxicity grading for the events will be presented. Events that lead to withdrawal will be identified in both the listing and summary tables. Serious adverse events will be summarized (by system, severity, and relationship to study medication) and narratives for all events will be reported.

### Vital Signs

Summary tables of all vital signs over time will be presented by shift tables and statistically derived. No inferential statistics will be performed.

### Biochemistry, Hematology, and Urinalysis

Laboratory parameters will be categorized by renal, hepatic, hematologic, biochemical and urinalysis. Descriptive statistics will be provided for all laboratory parameters over time in a summary table and trends will be discussed. Shift tables will be constructed to analyze shift from baseline to the minimum observed concentration, shift to the maximum observed concentration, and shift from baseline to the last observed concentration. Frequency of changes will be discussed.

### Toxicity Data

Toxicity data will be presented by severity for each dose cohort in each dosing ARM. The incidence of treatment discontinuations will also be summarized for each dose group. If there are sufficient numbers of subjects in each dose group, chi-square analyses will be used to compare the dose groups with respect to the incidence of specific toxicities. 95% confidence intervals will be constructed for these toxic events.

### Evaluation of Efficacy

All summaries will be provided on the ITT population. Data will be reviewed by inspection or statistically where appropriate. Statistical evaluation of data will be performed to assist in decision making for future development. The lack of statistical power to predict efficacy will be acknowledged.

### CD4+ T-lymphocyte Concentrations

CD4+ T-lymphocyte concentrations in the peripheral blood will be summarized. Results will be statistically derived (mean, standard deviation). Data will be compared between dosing cohorts and treatment arms.

### Hematologic Reconstitution

Neutrophil, lymphocyte, and platelet reconstitution to normal levels will be summarized after TXA127 over each dosage cohort. Results will be statistically derived (mean, standard deviation). Data will be compared between dosage cohorts and treatment arms.

### Sample Size, Definition of Evaluability

### Sample Size

The maximum number of evaluable study subjects is 36. The minimum is 2 evaluable study subjects. (See Section 6.2.) However, it is believed that TXA127 will be well tolerated and that it is likely that the study will progress with only three (3) subjects in each cohort plus 6 additional subjects for the confirmation of MTD and biologic activity studies. This would result in an anticipated minimum of 18 evaluable subjects enrolled if the MTD is not reached early, plus any replacement subjects.

### Evaluability

A subject will be deemed evaluable for safety evaluation if they receive any of the study material, including only one dose or a partial dose of the study material. Subjects who do not return for one of the follow-up visits at days 7, 14 or 28 will be replaced unless they develop a grade 3 or 4 adverse event within this 30 days window.

We claim:

1. (canceled)

2. (canceled)

3. (canceled)

4. (canceled)

5. (canceled)

6. A method for treating a subject in need of improved immune system function, comprising administering to the subject at least 300 μg/kg/day A(1-7) to provide improved immune system function.

7. The method of claim 6, wherein the subject is suffering from a tumor.

8. The method of claim 6, wherein the subject is undergoing or scheduled to undergo chemotherapy.

9. The method of claim 6, wherein the subject is suffering from an infection.

10. The method of claim 6, wherein the subject is immunodeficient due to conditions selected from the group consisting of aging, malnutrition, drug addiction, alcoholism, genetic predisposition, antibiotic treatment, skin damage, diabetes, and acquired immune deficiency syndrome (AIDS).

11. The method of claim 6, wherein the subject is selected from the group consisting of health care workers, first responders to emergencies, and hospitalized patients.

12. The method of claim 6, wherein the subject is selected from the group consisting of partners of HIV infected individuals, sex trade workers, health care workers, and intravenous drug users.

13. A method for treating or limiting acquired immune deficiency syndrome (AIDS) development in an HIV infected patient, comprising administering to an HIV-infected patient an amount effective of A(1-7) to treat or limit AIDS development in the patient.

14. The method of claim 13 wherein the HIV infected patient is undergoing highly active antiretroviral therapy.

15. The method of claim 13 wherein the HIV-infected patient has a CD4+ T-lymphocyte counts less than 200/mm3.


SEQUENCE LISTING (51 sequences)

SEQ ID NO: 1 - protein, 7 aa, Artificial
  Synthetic
DRVYIHP

SEQ ID NO: 2 - protein, 5 aa, Artificial
  Synthetic
DRVYI

SEQ ID NO: 3 - protein, 6 aa, Artificial
  Synthetic
DRVYIH

SEQ ID NO: 4 - protein, 8 aa, Artificial
  Synthetic
  MISC_FEATURE (1)..(1): Xaa is selected from the group consisting of H, Asp, Glu, Asn, Acpc, Ala, Me2Gly, Pro, Bet, Glu(NH2), Gly, Asp(NH2), and Suc, or is absent.
  MISC_FEATURE (2)..(2): Xaa is selected from the group consisting of Arg, Lys, Ala, Cit, Orn, Ser(Ac), Sar, D-Arg, and D-Lys.
  MISC_FEATURE (3)..(3): Xaa is selected from the group consisting of Val, Ala, Leu, norLeu, Ile, Gly, Lys, Pro, Aib, Acpc, and Tyr.
  MISC_FEATURE (4)..(4): Xaa is selected from the group consisting of Tyr, Tyr(PO3)2, Thr, Ser, homoSer, azaTyr, and Ala.
  MISC_FEATURE (5)..(5): Xaa is selected from the group consisting of Ile, Ala, Leu, norLeu, Val, and Gly.
  MISC_FEATURE (6)..(6): Xaa is selected from the group consisting of His, Arg, and 6-NH2-Phe.
  MISC_FEATURE (7)..(7): Xaa is selected from the group consisting of Pro and Ala.
  MISC_FEATURE (8)..(8): Xaa is selected from the group consisting of Phe, Phe(Br), Ile, Tyr, excluding sequences including Xaa4 as a terminal Tyr group
XXXXXXXX

SEQ ID NO: 5 - protein, 7 aa, Artificial
  Synthetic
RVYIHPF

SEQ ID NO: 6 - protein, 6 aa, Artificial
  Synthetic
VYIHPF

SEQ ID NO: 7 - protein, 7 aa, Artificial
  Synthetic
DRVYIHP

SEQ ID NO: 8 - protein, 6 aa, Artificial
  Synthetic
RVYIHP

SEQ ID NO: 9 - protein, 5 aa, Artificial
  Synthetic
VYIHP

SEQ ID NO: 10 - protein, 4 aa, Artificial
  Synthetic
IHPF

SEQ ID NO: 11 - protein, 6 aa, Artificial
  Synthetic
DRVYIH

SEQ ID NO: 12 - protein, 5 aa, Artificial
  Synthetic
DRVYI

SEQ ID NO: 13 - protein, 4 aa, Artificial
  Synthetic
DRVY

SEQ ID NO: 14 - skipped

SEQ ID NO: 15 - protein, 7 aa, Artificial
  Synthetic
  MOD_RES (2)..(2): Nle
RLYIHPF

SEQ ID NO: 16 - protein, 7 aa, Artificial
  Synthetic
  MOD_RES (4)..(4): Nle
RVYLHPF

SEQ ID NO: 17 - protein, 8 aa, Artificial
  Synthetic
DRPYIHPF

SEQ ID NO: 18 - skipped

SEQ ID NO: 19 - protein, 5 aa, Artificial
  Synthetic
YIHPF

SEQ ID NO: 20 - protein, 8 aa, Artificial
  Synthetic
DRVYVHPF

SEQ ID NO: 21 - protein, 8 aa, Artificial
  Synthetic
NRVYVHPF

SEQ ID NO: 22 - protein, 11 aa, Artificial
  Synthetic
APGDRIYVHPF

SEQ ID NO: 23 - protein, 8 aa, Artificial
  Synthetic
ERVYIHPF

SEQ ID NO: 24 - protein, 8 aa, Artificial
  Synthetic
DKVYIHPF

SEQ ID NO: 25 - protein, 8 aa, Artificial
  Synthetic
DRAYIHPF

SEQ ID NO: 26 - protein, 8 aa, Artificial
  Synthetic
DRVTIHPF

SEQ ID NO: 27 - protein, 8 aa, Artificial
  Synthetic
DRVYLHPF

SEQ ID NO: 28 - protein, 8 aa, Artificial
  Synthetic
DRVYIRPF

SEQ ID NO: 29 - protein, 8 aa, Artificial
  Synthetic
DRVYIHAF

SEQ ID NO: 30 - protein, 8 aa, Artificial
  Synthetic
DRVYIHPY

SEQ ID NO: 31 - protein, 8 aa, Artificial
  Synthetic
PRVYIHPF

SEQ ID NO: 32 - protein, 8 aa, Artificial
  Synthetic
DRPYIHPF

SEQ ID NO: 33 - protein, 8 aa, Artificial
  Synthetic
  MISC_FEATURE (4)..(4): Tyr is a Tyr(PO3)2
DRVYIHPF

SEQ ID NO: 34 - protein, 8 aa, Artificial
  Synthetic
  MOD_RES (3)..(3): Nle
DRLYIHPF

SEQ ID NO: 35 - protein, 8 aa, Artificial
  Synthetic
  MOD_RES (5)..(5): Nle
DRVYLHPF

SEQ ID NO: 36 - protein, 9 aa, Artificial
  Synthetic
  MISC_FEATURE (4)..(4): Ser is homoSer
DRVSYIHPF

SEQ ID NO: 37 - protein, 7 aa, Artificial
  Synthetic
  MISC_FEATURE (1)..(1): Xaa is selected from the group consisting of H, Arg, Lys, Ala, Orn, Citron, Ser(Ac), Sar, D-Arg, and D-Lys.
  MISC_FEATURE (2)..(2): Xaa is selected from the group consisting of Val, Ala, Leu, Nle, Ile, Gly, Lys, Pro, Aib, Acpc, and Tyr.
  MISC_FEATURE (3)..(3): Xaa is selected from the group consisting of Tyr, Tyr(PO3)2, Thr, Ser, homoSer, azaTyr, and Ala.
  MISC_FEATURE (4)..(4): Xaa is selected from the group consisting of Ile, Ala, Leu, Nle, Val, and Gly.
  MISC_FEATURE (5)..(5): Xaa is selected from the group consisting of His, Arg, and 6-NH2-Phe.
  MISC_FEATURE (6)..(6): Xaa is selected from the group consisting of Pro and Ala.
  MISC_FEATURE (7)..(7): Xaa is selected from the group consisting of Phe, Phe(Br), Ile, Tyr, excluding sequences including Xaa3 as a terminal Tyr group.
XXXXXXX

SEQ ID NO: 38 - protein, 7 aa, Artificial
  Synthetic
DRVAIHP

SEQ ID NO: 39 - protein, 7 aa, Artificial
  Synthetic
DRPYIHP

SEQ ID NO: 40 - protein, 7 aa, Artificial
  Synthetic
DRKYIHP

SEQ ID NO: 41 - protein, 7 aa, Artificial
  Synthetic
  MOD_RES (3)..(3): Nle
DRLYIHP

SEQ ID NO: 42 - protein, 8 aa, Artificial
  Synthetic
DRVYIHPI

SEQ ID NO: 43 - protein, 7 aa, Artificial
  Synthetic
RVAIHPF

SEQ ID NO: 44 - protein, 7 aa, Artificial
  Synthetic
RVAIHPF

SEQ ID NO: 45 - protein, 8 aa, Artificial
  Synthetic
GRVYIHPF

SEQ ID NO: 46 - protein, 8 aa, Artificial
  Synthetic
  MOD_RES (4)..(4): Nle
RVYNLHPF

SEQ ID NO: 47 - protein, 8 aa, Artificial
  Synthetic
  MISC_FEATURE (3)..(3): Xaa is Acpc
DRXYIHPF

SEQ ID NO: 48 - protein, 8 aa, Artificial
  Synthetic
  MISC_FEATURE (2)..(2): Xaa is Orn
DXVYIHPF

SEQ ID NO: 49 - protein, 8 aa, Artificial
  Synthetic
  MISC_FEATURE (2)..(2): Xaa is Citron
DXVYIHPF

SEQ ID NO: 50 - protein, 7 aa, Artificial
  Synthetic
  MISC_FEATURE (4)..(4): Tyr is Tyr(PO3)2
DRPAIHP

SEQ ID NO: 51 - protein, 7 aa, Artificial
  Synthetic
  MISC_FEATURE (3)..(3): Pro is a hydroxyPro
DRPAIHP